(19) United States
(12) Patent Application Publication
(10) Pub. No.: US 2022/0369665 A1
(43) Pub. Date: Nov. 24, 2022

(54) Generation of Cell-Based Products for Human Consumption

(71) Applicant: Upside Foods, Inc., Berkeley, CA (US)

(72) Inventors: Rachel Anne Porras VALENZUELA, Berkeley, CA (US); Neha ARORA, Emeryville, CA (US)

(21) Appl. No.: 17/661,014

(22) Filed: Apr. 27, 2022

Related U.S. Application Data
(60) Provisional application No. 63/180,828, filed on Apr. 28, 2021.

(51) Int. Cl. A23J 3/22; C12N 5/077; A23L 13/40

(52) U.S. Cl. CPC A23J 3/227; C12N 5/0656; C12N 5/0658; A23L 13/43; C12N 2510/02

(57) ABSTRACT

The present disclosure relates to methods of preparing cell-based products for human consumption, in particular, from populations of such cell types as hepatocytes, adipocytes, myoblasts, and/or fibroblasts.

### PRIORITY

This application claims priority to U.S. Provisional Application No. 63/180,828, filed Apr. 28, 2021, the entire content of which is incorporated herein by reference.

### FIELD

This invention is in the field of cell-based products for human consumption, in particular, products prepared from populations of cell types including hepatocytes, adipocytes, myoblasts, and/or fibroblasts. The present disclosure relates to novel consumable products and methods of preparing such consumable products.

### BACKGROUND

As the world's population continues to grow, the need for products for consumption is greater than ever. Given the expanding population, the market of conventional consumable products is struggling to meet the demand. In vitro produced cell-based products for consumption have emerged as an attractive option to supplement the demand for conventional products. Moreover, in vitro produced cell-based products help alleviate several drawbacks linked to conventional products. For instance, conventional meat products are associated with the controversial process of animal slaughter, increased microbial contamination, and such environmental concerns as poor conversion of caloric inputs, greenhouse gas emissions, and pollution.

Thus, it is an object of the invention to provide methods of preparing in vitro produced cell-based products for consumption. In particular, such cell-based products will be generated from populations of hepatocytes, adipocytes, myoblasts, and/or fibroblasts. Cell-based consumption products prepared from populations of hepatocytes, adipocytes, myoblasts, and/or fibroblasts may elicit a number of benefits such as, for example, discouraging animal slaughter and mistreatment, reducing environmental impact associated with raising animals, and eliminating the risk of contamination associated with slaughter. In addition, preparation of cell-based consumption products from such cell populations allows manufacturers to vary the fat content of such products, enabling control of such important consumer-desired characteristics as flavor, palatability, health, tenderness, and juiciness.

### SUMMARY

This invention generally relates to methods of preparing in vitro produced cell-based products for consumption from populations of such cell types as hepatocytes, adipocytes, myoblasts, and/or fibroblasts. By way of example, the cell-based products may be meat products, such as foie gras.

In a first embodiment, cell-based products for consumption may be prepared from populations of hepatocytes. In preferred embodiments, a prepared product may be foie gras and the populations of hepatocytes employed to generate the foie gras may exhibit steatosis, in particular, by accumulation of lipid droplets in the cytoplasm.

In a second embodiment, cell-based products for consumption may be prepared from populations of adipocytes, myoblasts, and/or fibroblasts. In preferred embodiments, a prepared product may be meat and the populations of cells employed to generate the meat may exhibit steatosis, in particular, by accumulation of lipid droplets in the cytoplasm.

### DESCRIPTION OF DRAWINGS

This patent or application file contains at least one drawing prepared in color. Copies of this patent or patent application publication with color drawing(s) will be provided by the Office upon request and payment of the necessary fee.

FIG. 1 depicts an exemplary flowchart demonstrating how chicken fibroblast can be differentiated to form hepatocytes or adipocytes to form a cell-based foie gras or cell-based meat or flavoring product.

FIG. 2, Images A and B, demonstrate fibroblasts in fibroblast media unable to transdifferentiate into adipocytes, either without oleic acid (FIG. 2, Image A) or with (FIG. 2, Image B) oleic acid. The fibroblast media consisted of 10% FBS, 2% chicken serum, and 100 μg/mL FGF2. FIG. 2, Images C and D, demonstrate transdifferentiation of fibroblasts into adipocytes in adipocyte differentiation media, both when oleic acid was not present (FIG. 2, Image C) and in the presence of the known inducer of lipid accumulation, oleic acid (FIG. 2, Image D). The adipocyte differentiation media consisted of DMEM-high glucose, supplemented with 10% FBS, 1% L-glut, 1 μM dexamethasone, 1 μM indomethacin, 500 μM 3-isobutyl-1-methylxantine (IBMX), and 10 μg/mL insulin. PPARgamma was overexpressed in all of FIG. 2, Images A-D.

FIG. 3 depicts quantitative analysis of mRNA expression levels of C/EBPalpha, FABP4, PPARgamma, and SREBP1 in adipocytes harvested six days after being transdifferentiated from chicken embryonic fibroblasts. Analysis was performed using qPCR in chicken embryonic fibroblasts (1) administered a PPARgamma vector and grown in fibroblast media (gray, striped), (2) administered a PPARgamma vector and grown in adipocyte differentiation media (checkered), (3) administered a PPARgamma vector and grown in fibroblast media in the presence of oleic acid (backslashes), (4) administered a PPARgamma vector and grown in adipocyte differentiation media in the presence of oleic acid (gray, solid), and (5) transfected with an empty, control vector (black). Gene expression was expressed as log₂ fold change.

FIG. 4, Images A-D, depict staining of Oil Red O in chicken embryonic fibroblasts overexpressing PPARgamma. Fibroblasts were fixed with 10% formalin and stained with Oil Red O in 60% isopropanol and hematoxylin to assess the presence of lipid droplets. Lipid droplets were stained red with Oil Red O, and cell nuclei were stained purple with hematoxylin. FIG. 4, Images A and B, depict fibroblasts in fibroblast media unable to form lipid droplets, either without oleic acid (FIG. 4, Image A) or with (FIG. 4, Image B) oleic acid. FIG. 4, Images C and D, depict fibroblasts transdifferentiated into adipocytes when grown in adipocyte differentiation media. Transdifferentiated adipocytes generated lipid droplets.

FIG. 5, Image A, depicts chicken embryonic fibroblasts transfected with an empty vector retaining fibroblast multipolar morphology and not exhibiting lipid droplets. FIG. 5, Image B, depicts chicken embryonic fibroblasts overexpressing C/EBPalpha and exhibiting lipid droplets phenotypic of adipocytes. Cells were grown in DMEM-F12 media with 10% FBS, 2% chicken serum, and 100 μg/mL FGF₂.

FIG. 6, Images A and B, depict staining of Oil Red O in chicken embryonic fibroblasts overexpressing C/EBPalpha and exhibiting the presence of lipid droplets. FIG. 6, Images C and D, depict staining of Oil Red O in control chicken embryonic fibroblasts. The fibroblasts were fixed with 10% formalin and stained with Oil Red O in 60% isopropanol and hematoxylin to assess the presence of lipid droplets. The lipid droplets were stained red with Oil Red 0, and the cell nuclei were stained purple with hematoxylin.

FIG. 7, Images A and B, depict DAPI staining of nuclei in chicken embryonic fibroblasts stained with Oil Red O that overexpress C/EBPalpha and exhibit lipid droplets. FIG. 7, Images C and D, depict DAPI and Oil Red O staining of control chicken embryonic fibroblasts. Nuclei were stained with DAPI at a 1:800 ratio.

FIG. 8, Image A, depicts control fibroblasts containing an empty vector. The control fibroblasts did not exhibit the presence of lipid droplets and retained a fibroblast multipolar morphology. FIG. 8, Image B, depicts the presence of lipid droplets in immortalized chicken embryonic fibroblasts that overexpress C/EBPalpha, which is indicative of transdifferentiation of the fibroblasts into adipocytes. Despite accumulation of lipid droplets, the transdifferentiated adipocytes continued to expand in population. The growth media for transdifferentiation comprised of DMEM-F12 with 10% FBS, 2% chicken serum, and 100 ug/ml FGF₂.

FIG. 9, Image A, demonstrates MyoD-overexpressing chicken embryonic myoblasts in myoblast growth media unable to transdifferentiate into adipocytes despite the presence of 500 μM of oleic acid. FIG. 9, Image B, demonstrates transdifferentiation of MyoD-overexpressing chicken embryonic myoblasts into adipocytes in adipocyte differentiation media in the presence of 500 μM of the known lipid accumulator oleic acid. The myoblast growth media comprised of DMEM-F12 with 20% FBS, 2% chicken serum, and 100 ug/ml FGF₂.

FIG. 10, Images A and D, demonstrate immortalized chicken embryonic myoblasts in myoblast growth media with MyoD overexpression (FIG. 10, Image D) and without (FIG. 10, Image A) MyoD overexpression. In FIG. 10, Images A and D, cells retained myoblast morphology and did not transdifferentiate into adipocytes. FIG. 10, Images B and E, demonstrate immortalized chicken embryonic myoblasts in myoblast growth media in the presence of 500 μM of the known lipid accumulator oleic acid and with MyoD overexpression (FIG. 10, Image E) and without (FIG. 10, Image B) MyoD overexpression. In FIG. 10, Images B and E, cells retained myoblast morphology and did not transdifferentiate into adipocytes. FIG. 10, Images C and F, demonstrate immortalized chicken embryonic myoblasts in adipocyte differentiation media in the presence of 500 μM of the known lipid accumulator oleic acid and with (FIG. 10, Image F) and without (FIG. 10, Image C) MyoD overexpression. In FIG. 10, Images C and F, cells were transdifferentiated into adipocytes, with more robust transdifferentiation shown in cells that overexpress MyoD (FIG. 10, Image F).

FIG. 11, Image A, depicts chicken embryonic fibroblasts transfected with a vector expressing both C/EBPalpha and GFP. Cells were selected under 1 μg/mL puromycin. FIG. 11, Image B, depicts a bright field view of chicken embryonic fibroblasts transfected with C/EBPalpha vector showing lipid formation. FIG. 11, Images C and D, depict GFP and bright field views of chicken embryonic fibroblasts transfected with a control, empty vector.

FIG. 12 depicts quantitative analysis of mRNA expression levels of C/EBPalpha, FABP4, PPARgamma, and SREBP1 in adipocytes harvested six days after being transdifferentiated from chicken embryonic fibroblasts. Analysis was performed using qPCR in chicken embryonic fibroblasts (1) administered a C/EBPalpha vector (backslashes), (2) transfected with an empty control vector (gray), or (3) not transfected (black). Gene expression was expressed as log₂ fold change.

FIG. 13, Image A, depicts control chicken embryonic fibroblasts containing an empty vector. Control cells retained a fibroblast multipolar morphology. FIG. 13, Image B, depicts transdifferentiation of immortalized chicken embryonic fibroblasts that overexpress HNF4alpha into cells that exhibit hepatocyte morphology ten days post-transfection with a vector.

FIG. 14 depicts quantitative analysis of mRNA expression levels of HNF4alpha, C/EBPalpha, and CYP3A4 in hepatocytes harvested six days after being transdifferentiated from chicken embryonic fibroblasts. Analysis was performed using qPCR in chicken embryonic fibroblasts (1) administered a P8 HNF4alpha vector (gray), (2) administered a P14 HNF4alpha vector (backslashes), (3) administered a P18 HNF4alpha vector (checkered), and (4) transfected with an empty, control vector (black). Gene expression was expressed as log 2 fold change. P=passage number.

FIG. 15, Images A and B, depict lipid accumulation both at 4× magnification (FIG. 15, Image A) and 10× magnification (FIG. 15, Image B) in chicken fibroblasts overexpressing C/EBPalpha and being grown in BR7 bioreactors. The transdifferentiated fibroblasts exhibited 60-80% confluence and importantly retained the adipocyte phenotype of lipid accumulation along with continued proliferation capacity even when scaled up from well plates as illustrated here in BR7 bioreactors.

FIG. 16 depicts lipid accumulation both at 4× magnification (FIG. 16, Image A) and 10× magnification (FIG. 16, Image B) in liver chicken fibroblasts overexpressing HNF4alpha and transdifferentiated into hepatocytes and being grown in BR7 bioreactors. This verifies that fibroblast transdifferentiated into hepatocytes according to the present methods maintain proliferative capacity and phenotype stability even when scaled up from well plates to BR7 roller bottles.

FIG. 17 depicts cell culture analysis in regards to metabolites (FIG. 17, Graph A) and pH (FIG. 17, Graph B) data from a control, non-transfected chicken fibroblasts grown in a large-scale production run.

FIG. 18 depicts cell culture analysis in regards to metabolites (FIG. 18, Graph A) and pH (FIG. 18, Graph B) data from chicken liver cells having HNF4alpha-overexpressed as grown in a large-scale production run.

FIG. 19 depicts the percent composition of fatty acids in tissue derived from hepatocyte-like cells (gray, slashes) versus control, non-transfected fibroblast tissue (black).

FIG. 20 depicts a chicken pate prototype developed from HNF4alpha tissue.

FIG. 21 depicts primary duck hepatocytes from juvenile Peking duck showcasing intracellular lipid accumulation.

FIG. 22 depicts hepatocytes derived from embryonic duck showcasing intracellular lipid accumulation.

FIG. 23, Graphs A-D, graphically illustrate protein levels (FIG. 23, Graphs A and B), moisture content (FIG. 23, Graph C), and pH (FIG. 23, Graph D) in fibroblast versus liver tissue for untransfected and HNF4alpha-transfected samples.

FIGS. 24 and 25 graphically illustrate a quantitative analysis of variations in cell culture media composition as ggCEBPa overexpressing cells grow and proliferate as a function of time.

FIG. 26 graphically illustrates mRNA expression data for passage 52 ggCEBPa cells.

FIG. 27 illustrates two separate cell passaging techniques and the resulting cell viability percentage from each technique.

### DETAILED DESCRIPTION OF THE INVENTION

Provided herein are methods and compositions related to the in vitro production of cell-based products for consumption comprising hepatocytes, adipocytes, myoblasts, and/or fibroblasts. For further detail, please reference U.S. application Ser. No. 17/033,635, the entire content of which is incorporated herein.

Before describing particular embodiments in detail, it is to be understood that the disclosure is not limited to the particular embodiments described herein, which can vary. It is also to be understood that the terminology used herein is for the purpose of describing particular illustrative embodiments only and is not intended to be limiting unless otherwise defined. The terms used in this specification generally have their ordinary meaning in the art, within the context of this disclosure and in the specific context where each term is used. Certain terms are discussed below or elsewhere in the specification, to provide additional guidance to the practitioner in describing the compositions and methods of the invention and how to make and use them. The scope and meaning of any use of a term will be apparent from the specific context in which the term is used. As such, the definitions set forth herein are intended to provide illustrative guidance in ascertaining particular embodiments of the invention, without limitation to particular compositions or biological systems.

As used in the present disclosure and the appended claims, the singular forms “a,” “an” and “the” include plural references unless the content clearly dictates otherwise.

Throughout the present disclosure and the appended claims, unless the context requires otherwise, the word “comprise”, or variations such as “comprises” or “comprising”, will be understood to imply the inclusion of a stated element or group of elements but not the exclusion of any other element or group of elements.

Unless specific definitions are provided, the nomenclature utilized in connection with, and the laboratory procedures and techniques of, molecular biology, cell biology, analytical chemistry, and synthetic organic chemistry described herein are those well-known and commonly used in the art. Standard techniques may be used for recombinant technology, molecular, biological, microbiological, chemical syntheses, and chemical analyses.

### Generation of Cell-Based Products for Consumption

Provided herein are methods to produce in vitro cell-based products for consumption.

### Cells

The cell-based products for consumption of the disclosure are compositions produced by the in vitro culturing of naturally occurring, transgenic, or modified animal cells in culture.

The cells used in the methods of the present disclosure can be primary cells, or cell lines. The methods provided herein are applicable to any metazoan cell in culture. Generally, the cells are from any metazoan species whose tissues are suitable for dietary consumption and demonstrate the capacity for skeletal muscle tissue specification.

In some embodiments, the cells are derived from any non-human animal species intended for human or non-human dietary consumption (e.g., cells of avian, ovine, caprine, porcine, bovine, piscine origin) (e.g., cells of livestock, poultry, avian, game, or aquatic species).

In some embodiments, the cells are from livestock such as domestic cattle, pigs, sheep, goats, camels, water buffalo, rabbits, and the like. In some embodiments, the cells are from poultry such as domestic chicken, turkeys, ducks, geese, pigeons, and the like. In some embodiments, the cells are from game species such as wild deer, gallinaceous fowl, waterfowl, hare, and the like. In some embodiments, the cells are from aquatic species or semi-aquatic species harvested commercially from wild fisheries or aquaculture operations, or for sport, including certain fish, crustaceans, mollusks, cephalopods, cetaceans, crocodilians, turtles, frogs and the like.

In some embodiments, the cells are from exotic, conserved or extinct animal species. In some embodiments, the cells are from Gallus gallus, Gallus domesticus, Bos taurus, Sous scrofa, Meleagris gallopavo, Anas platyrynchos, Salmo salar, Thunnus thynnus, Ovis aries, Coturnix, Capra aegagrus hircus, or Homarus americanus.

In some embodiments, the cells are primary stem cells, self-renewing stem cells, embryonic stem cells, pluripotent stem cells, induced pluripotent stem cells, or transdifferentiated pluripotent stem cells.

In some embodiments, the cells are modifiable by a genetic switch to induce rapid and efficient conversion of the cells to skeletal muscle for cultured production.

In some embodiments, the cells are myogenic cells, destined to become muscle, or muscle-like cells. In some embodiments, the myogenic cells are natively myogenic, e.g., myoblasts. Natively myogenic cells include, but are not limited to, myoblasts, myocytes, satellite cells, side population cells, muscle derived stem cells, mesenchymal stem cells, myogenic pericytes, or mesangioblasts.

In some embodiments, cells are of the skeletal muscle lineage. Cells of the skeletal muscle lineage include myoblasts, myocytes, and skeletal muscle progenitor cells, also called myogenic progenitors that include satellite cells, side population cells, muscle derived stem cells, mesenchymal stem cells, myogenic pericytes, and mesoangioblasts.

In some embodiments, the cells are non-myogenic, and such non-myogenic cells can be programmed to be myogenic, for example the cells may comprise fibroblasts modified to express one or more myogenic transcription factors. In exemplary embodiments, the myogenic transcription factors include MYOD1, MYOG, MYF5, MYF6, PAX3, PAX7, paralogs, orthologs, and genetic variants thereof. In some embodiments, the cells are native hepatocytes or stem cells. In some embodiments, the cells are modified to express one or more myogenic transcription factors as described in a PCT publication, WO/2015/066377, incorporated by reference herein in its entirety.

In some embodiments, the cells comprise a mixture of cell populations described herein, e.g., a mixture of fibrogenic cells and myogenic cells in co-culture, e.g., a mixture of fibroblasts and myoblasts in co-culture. In some embodiments, the cells used for the in vitro production of cell-based products for consumption are a mixture of fibroblasts and myoblasts in a suspension co-culture. In some embodiments the cells used for the in vitro production of cell-based products for consumption are a mixture of fibroblasts and myoblasts in an adherent co-culture. In some embodiments, the co-culture can further comprise adipocytes.

In some embodiments, the cells are in either a suspension culture or adherent co-culture, and comprise a mixture of fibroblasts and myoblasts, wherein the ratio of the fibroblasts to myoblasts (designated as F and M) ranges from about 5F:95M to about 95F:5M. In exemplary embodiments, the ratio of the fibroblasts to myoblasts is about 5F:95M, 10F:90M, 15F:85M, 20F:80M, 25F:75M, 30F:70M, 35F:65M, 40F:60M, 45F:55M, 50F:50M, 55F:45M, 60F:40M, 65F:35M, 70F:30M, 75F:25M, 80F:20M, 85F:15M, 90F:10M, or even about 95F:5M.

In some embodiments, the cells are genetically modified to inhibit a pathway, e.g., the HIPPO signaling pathway. Exemplary methods to inhibit the HIPPO signaling pathway as described in a PCT Application No. PCT/US2018/031276, incorporated by reference herein in its entirety.

In some embodiments, the cells are modified to express telomerase reverse transcriptase (TERT) and/or inhibit cyclin-dependent kinase inhibitors (CKI). In some embodiments, the cells are modified to express TERT and/or inhibit cyclin-dependent kinase inhibitors as described in a PCT publication, WO 2017/124100, incorporated by reference herein in its entirety.

In some embodiments, the cells are modified to express glutamine synthetase (GS), insulin-like growth factor (IGF), and/or albumin. Exemplary methods of modifying cells to express GS, IGF, and/or albumin are described in a PCT Application No. PCT/US2018/042187 which is incorporated by reference herein in its entirety.

In some embodiments, the cells may comprise any combinations of the modifications described herein.

### Cultivation Infrastructure

As referred to herein, a cultivation infrastructure refers to the environment in which the cells are cultured or cultivated to provide a two-dimensional or three-dimensional product for consumption.

A cultivation infrastructure may be a roller bottle, a tube, a cylinder, a flask, a petri-dish, a multi-well plate, a dish, a vat, an incubator, a bioreactor, an industrial fermenter, and the like.

While the cultivation infrastructure itself may have a three-dimensional structure or shape, the cells cultured in the cultivation infrastructure may form a monolayer of cells or a multilayer of cells. Compositions and methods of the present disclosure can promote a three-dimensional growth of metazoan cells in the cultivation infrastructure to provide a scaffold-less self-assembly of a three-dimensional cellular biomass.

A three-dimensional cultivation infrastructure may be sculpted into different sizes, shapes, and forms, as desired, to provide the shape and form for the muscle cells to grow and resemble different types of muscle tissues such as steak, tenderloin, shank, chicken breast, drumstick, lamb chops, fish fillet, lobster tail, etc. The three-dimensional cultivation infrastructure may be made from natural or synthetic biomaterials that are non-toxic so that they may not be harmful if ingested. Natural biomaterials may include, for example, collagen, fibronectin, laminin, or other extracellular matrices. Synthetic biomaterials may include, for example, hydroxyapatite, alginate, polyglycolic acid, polylactic acid, or their copolymers. The three-dimensional cultivation infrastructure may be formed as a solid or semisolid support.

A cultivation infrastructure can be of any scale and support any volume of cellular biomass and culturing reagents. In some embodiments, the cultivation infrastructure ranges from about 10 μL to about 100,000 L. In exemplary embodiments, the cultivation infrastructure is about 10 μL, about 100 μL, about 1 mL, about 10 mL, about 100 mL, about 1 L, about 10 L, about 100 L, about 1000 L, about 10,000 L, or even about 100,000 L.

In some embodiments, the cultivation infrastructure comprises a substrate. A cultivation infrastructure may comprise a permeable substrate (e.g., permeable to physiological solutions) or an impermeable substrate (e.g., impermeable to physiological solutions). The substrate can be flat, concave, or convex. The substrate may be textured so as to promote cell growth and cell sheet attachment.

In some embodiments, the culturing of cells in the cultivation infrastructure can induce the production of extracellular matrix (ECM) that may act as an autologous scaffold to direct three-dimensional cellular growth, e.g., to direct attachment, proliferation, and hypertrophy of cells on a plane perpendicular to the substrate.

In some embodiments, the cultivation infrastructure may not comprise an exogenously added scaffold to promote self-assembly of a three-dimensional cellular biomass. In some embodiments, the cultivation infrastructure may not comprise exogenous scaffolds such as a hydrogel or soft agar.

### Culturing Conditions

The culturing conditions for the generation of cell-based products for consumption are generally aseptic, and sterile.

Cells can be grown in an adherent culture format to form a cell sheet or can be grown in a suspension culture format to form a cell pellet. Table 1 provides exemplary culture methods for the various products that can be produced in vitro.

TABLE 1
Cell culture methods used to generate in vitro produced cell-based meat

| | | Culture | | |
|---|---|---|---|---|
| | | Condition | | |
| | Sample | Cell Type(s) | Culture | Base |
| Method # | ID | (ratio) | format | media |
| 1 | A. Platyrhynchos (duck) | Co-culture | Adherent | DMEM-F12 with |
| | fibroblast/myoblast tissue 1 | F/M (50/50) | | FBS (High), BS |
| | | | | (High), CS (Low), |
| | | | | HS (Low) |
| 2 | A. Platyrhynchos (duck) | Monoculture | Adherent | DMEM-F12 with |
| | fibroblast tissue 1 | F | | FBS (High), BS |
| | | | | (High), CS (Low), |
| | | | | HS (Low) |
| 3 | Bos (Cow) fibroblast | Monoculture | Adherent | DMEM-F12 with |
| | tissue 1 | F | | FBS (High), BS |
| | | | | (High), CS (Low), |
| | | | | HS (Low) |
| 4 | Gallus (chicken) fibroblast | Monoculture | Adherent | DMEM-F12 with |
| | tissue 1 | F | | FBS (High), CS |
| | | | | (Low) |
| 5 | Gallus (chicken) fibroblast | Monoculture | Adherent | DMEM-F12 with CS |
| | tissue 2 | F | | (High) BS (Low) |
| 6 | Gallus (chicken) fibroblast | Monoculture | Adherent | DMEM-F12 with CS |
| | tissue 3 | F | | (High) BS (High) |
| 7 | Gallus (chicken) fibroblast | Monoculture | Adherent | DMEM-F12 with BS |
| | tissue 4 | F | | (High), CS (Low) |
| 8 | Gallus (chicken) fibroblast | Monoculture | Adherent | DMEM-F12 with |
| | cells 1 | F | | 10% FBS |
| 9 | Gallus (chicken) | Co-culture | Adherent | DMEM-F12 with |
| | fibroblast/myoblast tissue 1 | F/M (30/70) | | FBS (High), CS |
| | | | | (Low) |
| 10 | Gallus (chicken) fibroblast | Monoculture | Adherent | DMEM-F12 with BS |
| | tissue 5 | F | | (High), CS (Low) |
| 11 | Gallus (chicken) myoblast | Monoculture | Suspension | DMEM-F12 with BS |
| | cells 1 | M | | (High), CS (Low) |
| 12 | Gallus (chicken) | Co-culture | Adherent | DMEM-F12 with BS |
| | fibroblast/myoblast tissue 2 | F/M (30/70) | | (High), CS (Low) |
| 13 | Gallus (chicken) | Co-culture | Adherent | DMEM-F12 with BS |
| | fibroblast/myoblast tissue 3 | F/M (50/50) | | (High), CS (Low) |
| 14 | Gallus (chicken) | Co-culture | Adherent | DMEM-F12 with BS |
| | fibroblast/myoblast tissue 4 | F/Monoclonal | | (High), CS (Low) |
| | | M (50/50) | | |
| 15 | Gallus (chicken) | Co-culture | Adherent | Chemically-defined |
| | fibroblast/myoblast tissue 5 | F/Monoclonal | | media with BS (low) |
| | | M (70/30) | | |
| 16 | Gallus (chicken) myoblast | Monoculture | Suspension | Chemically defined |
| | cells 2 | M | | media formula. No |
| | | | | serum |
| 17 | Gallus (chicken) myoblast | Monoculture | Suspension | SMEM-F12 with BS |
| | cells 3 | M | | (high), CS (low) |

In some embodiments, the media is substantially free of serum or other components derived from an animal.

Accordingly, an exemplary method of producing in vitro produced cell-based meat comprises: (a) providing fibroblasts and/or myoblasts from a non-human organism; (b) culturing the fibroblasts and/or myoblasts in media under conditions under which the fibroblasts and/or myoblasts grow in either suspension culture or adherent culture, wherein the media is substantially free of serum and other components derived from an animal.

In some embodiments, the cells are grown in a suspension culture, e.g., in a shake flask, and the product of the culture is centrifuged, yielding a cell pellet. In other embodiments, the cells are grown in adherent culture, and the product of the culture is a cell sheet.

### Formulation

The cell-based products for consumption of the disclosure may be processed into any variety of products including, but not limited to, cell-based meat products, foie gras, supplements, and vitamins. Exemplary cell-based products of the disclosure include cell-based meat products, such as, for example, avian meat products, chicken meat products, duck meat products, and bovine meat products.

### Characteristics of Cell-Based Products for Consumption

Provided herein are in vitro produced cell-based products for consumption comprising a number of unique features that allow them to be distinguished from conventional products (which can involve the slaughter or demise of live animals). The in vitro methods can also be tailored to achieve desired traits such as health and sensory benefits.

### Hormones

As compared to conventional products, the in vitro produced cell-based products of the disclosure comprise a significantly lower amount of steroid hormones. For example, using the in vitro culturing methods described, there need not be any exogenous hormones added into culture thus resulting in lower or non-existent hormonal levels in a resulting cell-based meat product. Accordingly, in some embodiments, the cell-based product is substantially free of steroid hormones (i.e., contains little or no steroid hormones). This is in contrast to the animals raised for conventional meat production, which are often fed or otherwise administered exogenous hormones.

Accordingly, in some embodiments, the cell-based product of the disclosure comprises no more than about 1 ug, 0.5 ug, 0.1 ug, 0.05 ug, 0.01 ug, 0.005 ug, or even about 0.001 ug steroid hormone/kg dry mass of cell-based product. In some embodiments, the cell-based product comprises no more than about 1 ug, 0.5 ug, 0.1 ug, 0.05 ug, 0.01 ug, 0.005 ug, or even about 0.001 ug progesterone/kg dry mass of cell-based product. In some embodiments, the cell-based product comprises no more than about 1 ug, 0.5 ug, 0.1 ug, 0.05 ug, 0.01 ug, 0.005 ug, or even about 0.001 ug testosterone/kg dry mass of cell-based product. In some embodiments, the cell-based product comprises no more than about 0.05 ug, 0.01 ug, 0.005 ug, or even about 0.001 ug estradiol/kg dry mass of cell-based product. In exemplary embodiments, the cell-based product comprises no more than about 35 ng estradiol/kg dry mass of cell-based product.

### Microbial Contamination

Using the sterile, laboratory-based in vitro culturing methods described, the cell-based product is substantially free of microbial contaminants. “Substantially free” means that the concentration of microbes or parasites is below a clinically significant level of contamination, i.e., below a level wherein ingestion would lead to disease or adverse health conditions. Such low levels of contamination allow for an increased shelf life. This is in contrast to animals raised for conventional meat production. As used herein, microbial contamination includes, but is not limited to, bacteria, fungi, viruses, prions, protozoa, and combinations thereof. Harmful microbes may include coliforms (fecal bacteria), E. coli, yeast, mold, Campylobacter, Salmonella, Listeria, and Staph.

In addition, cells grown in culture may be substantially free from parasites such as tapeworms that infect cells of whole animals and that are transferred to humans through consumption of insufficiently cooked meat.

### Antibiotics

Relative to conventional products, in vitro produced cell-based products of the disclosure comprise a significantly lower amount of antibiotics, or are substantially free of antibiotics, or are free of antibiotics entirely. For example, using the in vitro culturing methods described herein, the use of antibiotics in culture can be controlled or eliminated, thus resulting in lower or non-existent antibiotic levels in the resulting cell-based product. Accordingly, in some embodiments, the cell-based product is substantially free of antibiotics (i.e., contains little or no antibiotics). This is in contrast to animals raised for conventional meat production, which are often fed or otherwise administered exogenous antibiotics.

Accordingly, in some embodiments, the cell-based product of the disclosure comprises no more than about 100 ug antibiotics/kg dry mass of cell-based product, 90 ug antibiotics/kg dry mass of cell-based product, 80 ug antibiotics/kg dry mass of cell-based product, 70 ug antibiotics/kg dry mass of cell-based product, 60 ug antibiotics/kg dry mass of cell-based product, 50 ug antibiotics/kg dry mass of cell-based product, 40 ug antibiotics/kg dry mass of cell-based product, 30 ug antibiotics/kg dry mass of cell-based product, 20 ug antibiotics/kg dry mass of cell-based product, 10 ug antibiotics/kg dry mass of cell-based product, 5 ug antibiotics/kg dry mass of cell-based product, 1 ug antibiotics/kg dry mass of cell-based product, 0.5 ug antibiotics/kg dry mass of cell-based product, 0.1 ug antibiotics/kg dry mass of cell-based product, 0.05 ug antibiotics/kg dry mass of cell-based product, or even about 0.01 ug/kg of antibiotics/kg dry mass of cell-based product.

### Lipids

As compared to conventional products, the in vitro produced cell-based products of the disclosure comprise a lower average total lipid (fat) content. For example, cell-based meat generally has an average total fat content between about 0.5% to about 5.0%, whereas the fatty acid content in conventional meat varies widely and can range from about 3% to about 18%, depending on the cut of meat.

Accordingly, in some embodiments, the cell-based products of the disclosure comprise an average total fat content of about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0%, when measured as a % of total wet mass of the cell-based product. A lower fat content provides a lower caloric content, as well as other related health benefits, when as compared to conventional products.

The methods provided herein can alter specific fatty acid profiles to achieve desired flavor characteristics or fatty acid profiles. The lower levels of fatty acids in the cell-based products of the disclosure also promote an increased shelf life, for example by leading to lower levels of fatty oxidation in the products.

### Amino Acids

The cell-based meat products of the disclosure generally comprise about 50 g to about 95 g by weight of amino acids per 100 g dry mass.

### Vitamin E Content

As compared to conventional products, the in vitro produced cell-based products of the disclosure comprise a higher Vitamin E (aTocopherol) content. In some embodiments, the cell-based products of the disclosure comprise at least about 0.5 mg, at least about 0.6 mg, at least about 0.7 mg, at least about 0.8 mg, at least about 0.9 mg, or at least about 1.0 mg/Vitamin E/100 g wet mass of cell-based product.

### Moisture Content

The cell-based products of the disclosure generally have a moisture content of about 65% to about 95%.

### Architecture of Cell-Based Meat

By way of example, cell-based meat, unless otherwise manipulated to include, does not include vascular tissues, such as veins and arteries, whereas conventional meat does contain such vasculature, and contains the blood found in the vasculature. Accordingly, in some embodiments, the cell-based meat does not comprise any vasculature.

Likewise, cell-based meat, although composed of muscle or muscle-like tissues, unless otherwise manipulated to include, does not comprise functioning muscle tissue. Accordingly, in some embodiments, the cell-based meat does not comprise functioning muscle tissue.

It is noted that features such as vasculature and functional muscle tissue can be further engineered into the cell-based meat, should there be a desire to do so.

### Supplementation

In other embodiments, other nutrients, such as vitamins, may be added to increase the nutritional value of the cell-based product. For example, this may be achieved through the exogenous addition of the nutrients to the growth medium or through genetic engineering techniques.

### Shelf Life

A significant portion of meat and meat products are spoiled every year. It is estimated that approximately 3.5 billion kg of poultry and meat are wasted at the consumer, retailer and foodservice levels which have a substantial economic and environmental impact (Kantor et al. (1997)). A significant portion of this loss is due to microbial spoilage.

Conventional meat is perishable and has a relatively short shelf-life stability (interchangeably referred to as simply “shelf life” herein). The shelf life is the amount of time a food remains fit for human consumption. The composition of conventional meat and the conditions used to slaughter and harvest the meat create favorable growth conditions for various microorganisms including fecal bacteria (e.g., coliform bacteria). Meat is also very susceptible to spoilage due to chemical, oxidative and enzymatic activities. It is generally regarded that microbial growth, oxidation and enzymatic autolysis are three mechanisms responsible for the spoilage of meat. The breakdown of fat, protein, and carbohydrates of meat results in the development of off-odors and off-flavor and these the off-odors and off-flavors make the meat objectionable for human consumption. Depending on the species and method of harvest, conventional meat products are not safe to consume after a relatively short period of storage time. For example, chicken should be cooked within a few days of purchasing. Cooked poultry can be safely stored in the fridge for only 4 days and the freezer for up to 4 months. It is, therefore, necessary to control meat spoilage in order to increase its shelf life and maintain its nutritional value, texture, and flavor.

In vitro produced cell-based meat, through its method of production and composition, produces a meat product that has extended shelf life compared to conventional meat products and does not require the addition of preservative agents to obtain the shelf-life stability. The composition of cell-based meat is such that fewer off-odors and off-flavors are detected. In addition, the manufacturing methods used to produce in vitro cell-based meat require clean and aseptic conditions. These conditions ensure that microbial cell counts in both harvested products and subsequent food processing are low. These multiple factors contribute to extended shelf-life stability of in vitro cell-based meat.

The shelf life due to spoilage of the cell-based meat of the disclosure is enhanced relative to conventional meat. This is the case both at room temperature (about 25° C.) and at colder temperatures (e.g., about 4° C.). The increased shelf life is associated with reduced contamination, composition of the cell-based meat, reduced degradation of the cell-based meat and slower rates of change in color, spoilage, smell and flavor of the cell-based meat.

Without being bound to theory or mechanism, there is a decrease in total fatty acid content in the cell-based meat, as compared to conventional meat, resulting in lower levels of fatty acid oxidation, leading to slower rates of change in the color, smell, or flavor of the meat.

Without being bound to theory or mechanism, there is a decrease in the number of lipases in the cell-based meat, as compared to conventional meat, resulting in lower levels fatty acid breakdown, leading to slower rates of change in the color, smell, or flavor of the meat.

Without being bound to theory or mechanism, due to the absence of vasculature in the cell-based meat, when compared to conventional meat, there is less oxygen present, resulting in lower levels of fatty acid oxidation and the growth of aerobic bacteria, leading to reduced microbial contamination levels, and leading to slower rates of change in the color, smell, or flavor of the meat.

Without being bound to theory or mechanism, due to the absence of functional muscle tissue (e.g., myoglobin) in the cell-based meat when compared to conventional meat, there is less oxygen present, resulting in lower levels of fatty acid oxidation and the growth of aerobic bacteria, leading to reduced microbial contamination levels, and leading to slower rates of change in the color, smell, or flavor of the meat.

Without being bound to theory or mechanism, due to higher amounts of Vitamin E in the cell-based meat when compared to conventional meat, there are higher levels of antioxidant activity, resulting in protection against fatty acid oxidation, and leading to slower rates of change in the color, smell, or flavor of the meat.

Accordingly, in some embodiments, as compared to conventional meat, the shelf life of cell-based meat is increased by at least about 1.5×, at least about 2×, at least about 2.5×, at least about 3×, at least about 3.5×, at least about 4×, at least about 4.5×, at least about 5×, at least about 5.5×, at least about 6×, at least about 6.5×, at least about 7×, at least about 7.5×, at least about 8×, at least about 8.5×, at least about 9×, at least about 9.5×, or even at least about 10×. The shelf-life increases are observed both at about 4° C., and about 25° C., and all temperatures in between inclusive of the endpoints.

Cell-Based Products for Consumption Prepared from Such Cell Populations as Hepatocytes, Adipocytes, Myoblasts, and/or Fibroblasts

In preferred embodiments of the invention, the cell-based products for consumption may be prepared from such cell populations as hepatocytes, adipocytes, myoblasts, and/or fibroblasts. In a particular embodiment, the cell-based products for consumption may be cell-based meat or cell-based foie gras. By way of example, the cell-based meat products may be cell-based deep fried pork rinds (e.g., chicharrons) comprised of animal skin and fat. The cell-based foie gras may comprise pate and liver spread products, such as chicken liver pate, liver sauces, liver spread, and liver wurst. Alternatively, the cell-based products for consumption may be liver supplements or dog food. In certain embodiments, adipocytes may be employed as flavoring agents or products (e.g., dehydrated adipocytes) for cell-based meat products, plant-based meat products, and/or hybrid products, such as products comprising plants and cell-based meat. In addition, the “fattiness” of such products can be assessed by quantifying lipid droplets, submitting the quantifications for fatty acid analysis, and determining total lipid composition through lipidomics, including measurements involving mass spectrometry.

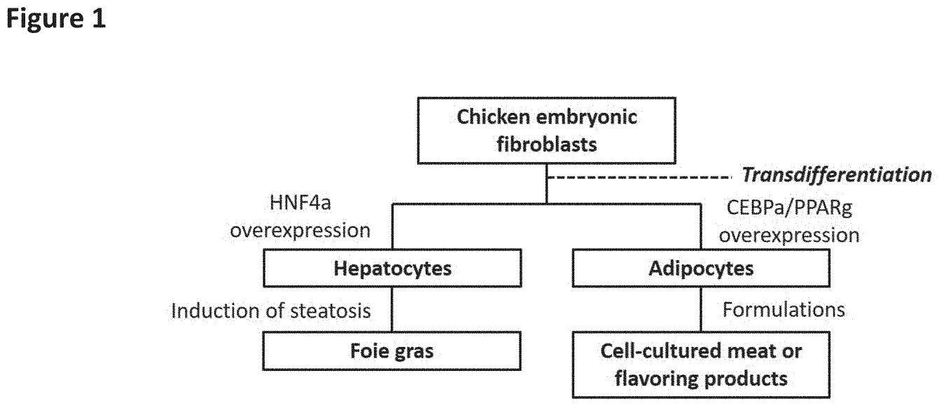

FIG. 1 depicts an exemplary flowchart outlining methods for transdifferentiating fibroblasts into cells having lipid accumulating phenotypes. For instance, chicken or duck embryonic fibroblast may be transdifferentiated into hepatocytes via overexpression of HNF4a. Inducing steatosis in these hepatocytes stimulates lipid accumulation to a degree that the resulting cells may be used to form a foie gras or pate food product. As an alternative, chicken or duck embryonic fibroblast may be transdifferentiated into adipocytes via overexpression of CEBPa, PPARg, or some combination thereof. These adipocytes may then be useful for production of a cell-cultured meat product or for flavoring such products.

In a preferred first embodiment, cell-based foie gras may be generated from a population of hepatocytes. In certain embodiments, the cell-based foie gras may comprise a mixture of hepatocytes and fibroblasts. Alternatively, the cell-based meat may comprise a single population of hepatocytes.

By way of example, primary hepatocytes may be procured from such animals as ducks, geese, or chickens. The procured primary hepatocytes may then be expanded and immortalized. In alternative embodiments, hepatocyte-like cells may be transdifferentiated from fibroblasts. For example, transdifferentiation of fibroblasts into hepatocyte-like cells may be accomplished by reprogramming such fibroblast genes as ATF5, PROX1, FOXA2, FOXA3, HNF4A, ONECUT1, NR1H4, MLXIPL, NR5A2, and XBP1. “Transdifferentiation” in the context of the present disclosure is defined as a process in which one mature, specialized cell type changes into a separate cell type without entering a pluripotent state. Transdifferentiation involves ectopic expression of transcription factors and/or other stimuli. Transdifferentiation may be used interchangeably with such terms as “lineage reprogramming” or “conversion”. For example, fibroblasts engineered to express adipocyte phenotypes, such as lipid accumulation, may be characterized as having been transdifferentiated into adipocytes, and likewise for hepatocytes. Transdifferentiated cells are subjected to a selection process in order to ensure full conversion of fibroblasts to hepatocytes. In preferred embodiments, the fibroblasts may be chicken or duck fibroblasts. In certain embodiments, the chicken or duck fibroblasts may be primary and immortalized. In other embodiments, pluripotent stem cells may serve as a source for hepatocytes.

In certain embodiments, the hepatocytes may be transfected to induce steatosis. By way of example, steatosis of hepatocytes may be induced by overexpression of transfected genes. In preferred embodiments, the overexpressed genes may be PPARgamma, C/EBPalpha, SREBP1, or SREPB2. Alternatively, steatosis of hepatocytes may be induced by downregulation of specific genes or by addition of oleic acid. In preferred embodiments, the downregulated genes may be OSR1, PRRX1, LHX9, TWIST2, or INSIG2. Transfection of hepatocytes may be accomplished by any suitable mechanism including, but not limited to, the cloning of genes to be overexpressed into a vector. In particular examples, the vector may be a PhiC31 vector or inducible vectors (e.g., tetracycline vectors and cumate vectors). Downregulation of genes may be accomplished by, for example, transfection of siRNA or CRISPR guide RNAs.

Once successfully transfected, steatosis of hepatocytes may be induced. Degree of hepatocyte steatosis may be determined by degree of lipid accumulation, including, but not limited to, number of lipid droplets formed. Hepatocytes exhibiting extensive steatosis may then be selected and expanded to generate foie gras.

In a preferred second embodiment, cell-based meat may be generated from populations of adipocytes, fibroblasts, and/or myoblasts. In certain embodiments, the cell-based meat may comprise a mixture of adipocytes, fibroblasts, and/or myoblasts. Alternatively, the cell-based meat may comprise a single population of adipocytes.

In certain embodiments, primary fibroblasts may be procured from such animals as chickens, ducks, geese, or other avian species. Alternatively, primary myoblasts or primary adipocytes may be procured from such animals. The procured primary fibroblasts, myoblasts, or adipocytes may then be expanded and immortalized. The immortalized fibroblasts or myoblasts may then be transfected to induce transdifferentiation into adipocytes or adipocyte-like cells. Additionally, transfection may induce steatosis in the resulting adipocytes or adipocyte-like cells. In preferred embodiments, the disclosed immortalized cell types retain differentiation capacity even after being cultured for 60 Population Doubling Levels (PDLs) or more. Cells having undergone so many population doublings are often referred to as ‘late passage’ cells. Late passage may be defined by at least 60 PDLs, at least 70, 80, 90, 100, 110, 120, or 130 passages. It should be noted that each individual passage, e.g. “passage number”, refers to 2 or more population doublings.

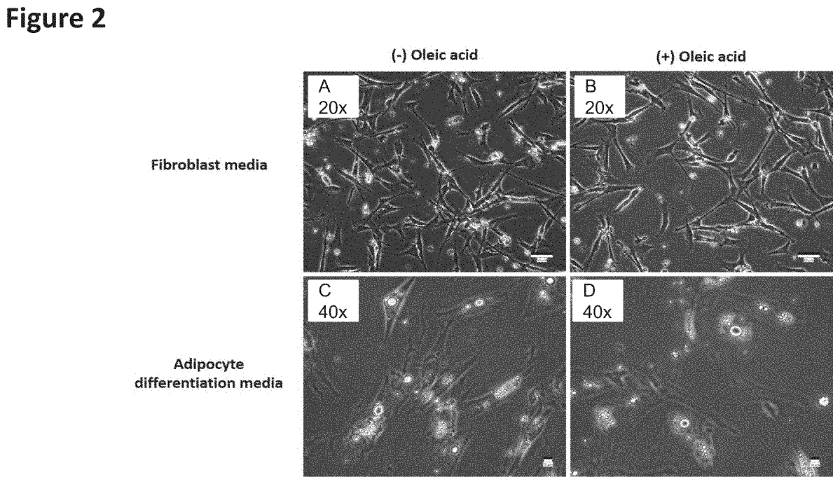

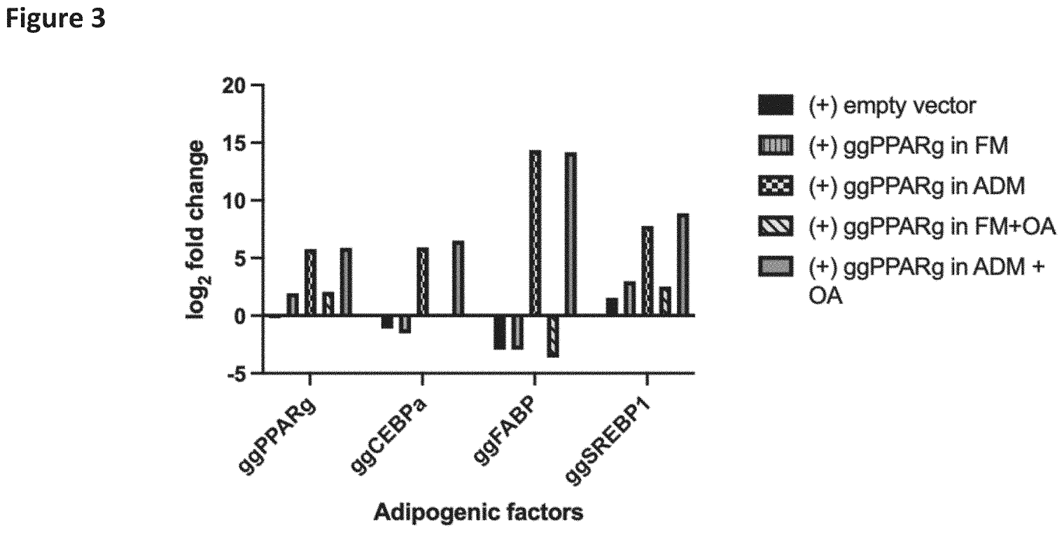

By way of example, transdifferentiation into adipocytes or adipocyte-like cells and steatosis in such cells may be induced by overexpression of transfected genes. Alternatively, transdifferentiation into adipocytes or adipocyte-like cells and steatosis may be induced by employing adipocyte differentiation media, as shown in FIG. 2, Images C and D, and FIG. 3 where fibroblasts overexpressing PPARgamma were transdifferentiated into adipocytes when grown in adipocyte differentiation media, both in the presence of oleic acid and when oleic acid was not present. Transdifferentiated cells may be subjected to a selection process in order to ensure full conversion to adipocytes. In other embodiments, primary adipocytes may be isolated from adipogenic tissues or tissues of mesenchymal origin. In preferred embodiments, the overexpressed genes may be PPARgamma, C/EBPalpha, C/EBPgamma, MyoD1, SREBP1, or SREPB2. Alternatively, steatosis of adipocytes or adipocyte-like cells may be induced by downregulation of specific genes. In preferred embodiments, the downregulated genes may be MyoD1, OSR1, PRRX1, LHX9, TWIST2, or INSIG2. In a separate preferred embodiment, upregulation of MyoD may induce transdifferentiation of myoblasts into adipocytes. Transfection of fibroblasts may be accomplished by any suitable mechanism including, but not limited to, the cloning of genes to be overexpressed into a vector. In particular examples, the vector may be a PhiC31 vector. Downregulation of genes may be accomplished by transfection of siRNA or CRISPR guide RNAs. In a particular example, MyoD1 may be downregulated in myoblasts via siRNA or CRISPR guide RNAs to initiate transdifferentiation of the myoblasts into adipocyte-like cells. In other embodiments, pluripotent stem cells may serve as a source for adipocytes.

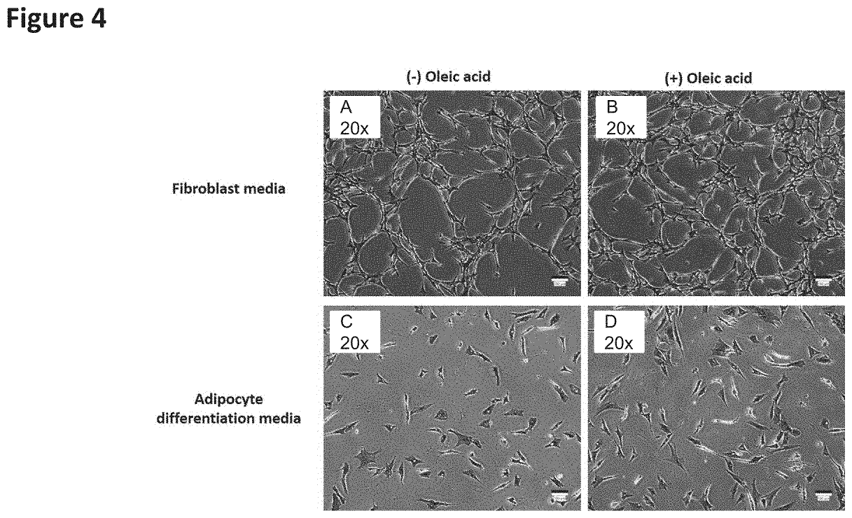

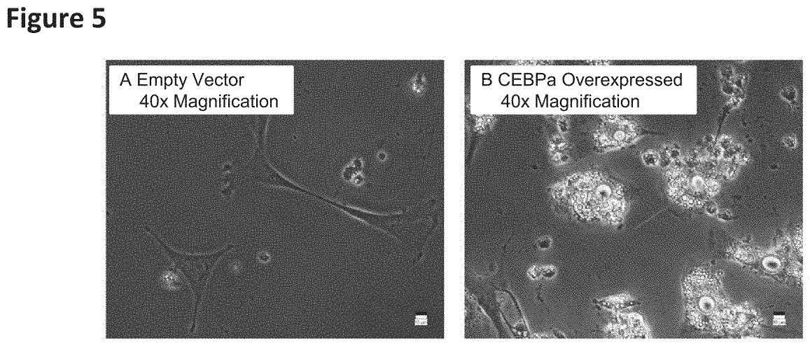

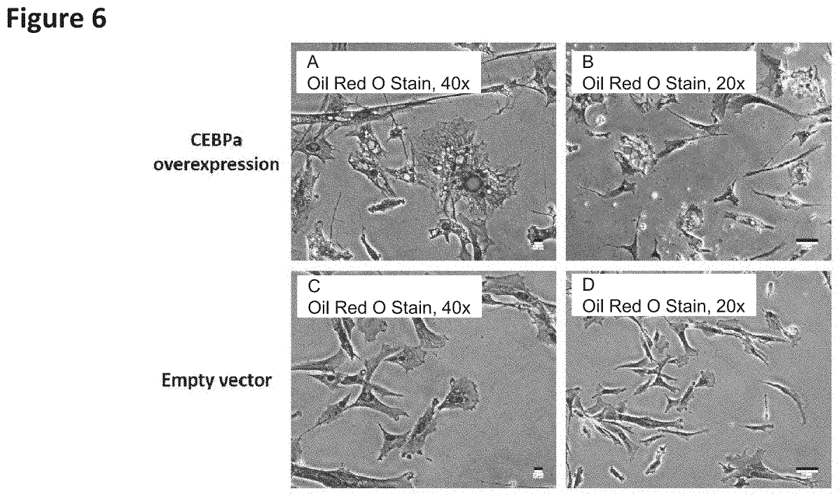

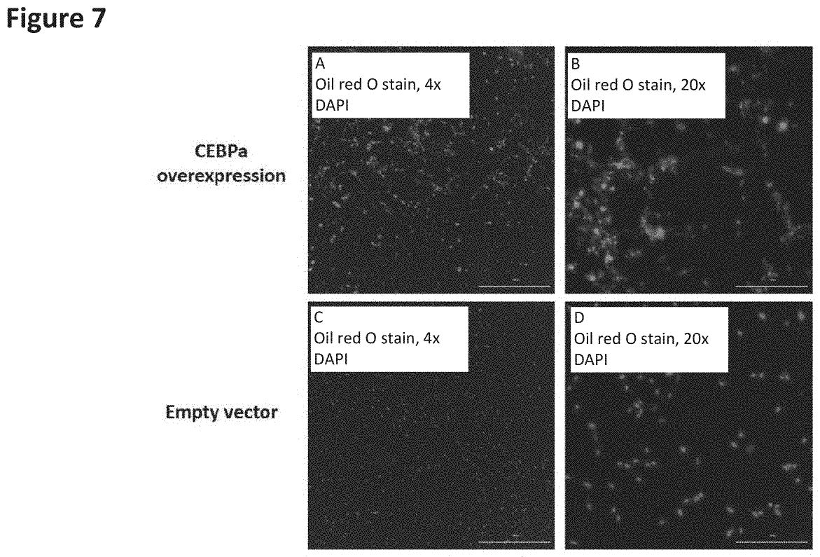

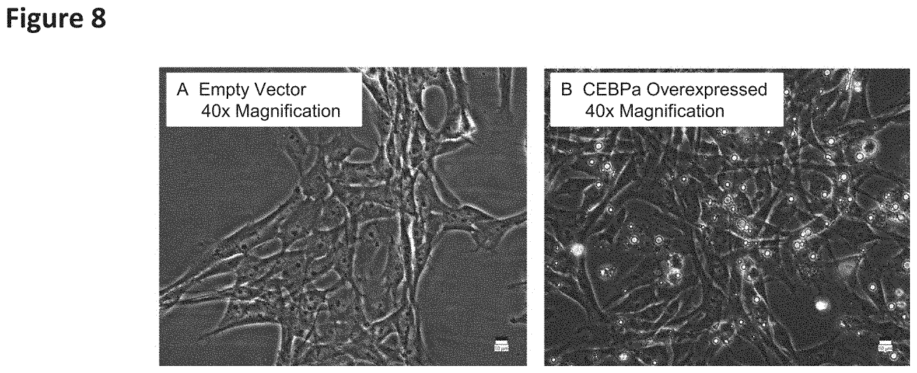

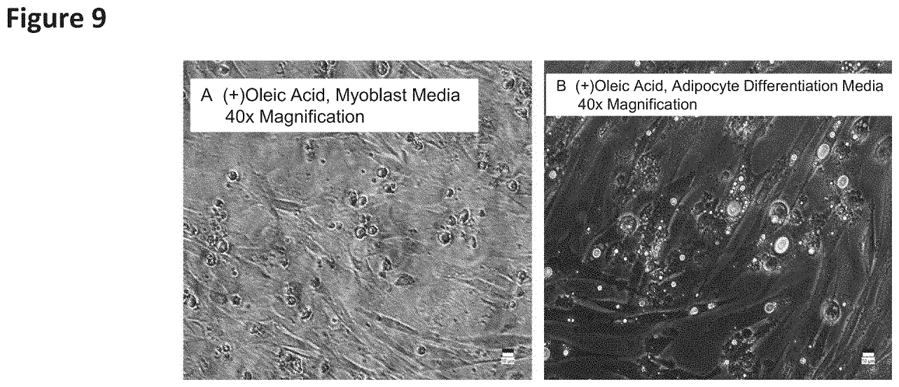

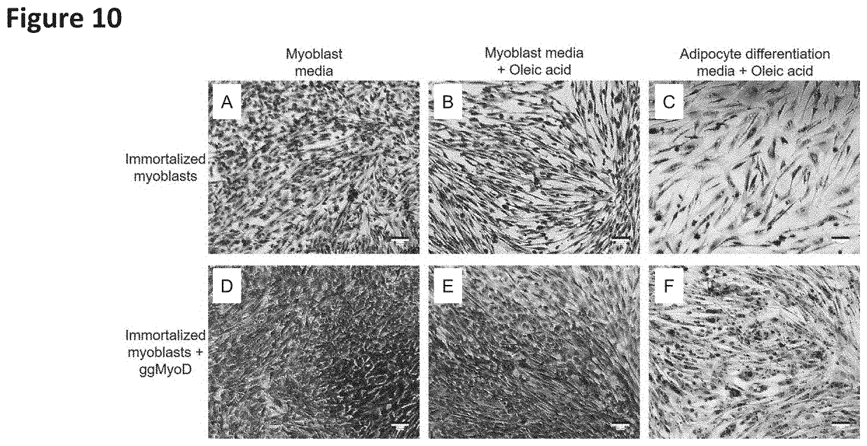

Once successfully transfected, transdifferentiation into adipocytes or adipocyte-like cells and steatosis in such cells may be induced. Degree of steatosis may be determined by a degree of lipid accumulation, including, but not limited to, a number of lipid droplets formed. Adipocytes or adipocyte-like cells exhibiting extensive steatosis may then be selected and expanded to generate meat. As shown in FIG. 4, Images C and D, fibroblasts overexpressing PPARgamma were transdifferentiated into adipocytes when grown in adipocyte differentiation media, both in the presence of oleic acid and when oleic acid was not present. Subsequently, the transdifferentiated adipocytes generated lipid droplets. In particular embodiments, overexpression of such genes as C/EBPalpha, C/EBPgamma, and MyoD in immortalized fibroblasts or myoblasts may surprisingly facilitate their proliferation and transdifferentiation into adipocytes, as well as steatosis of the transdifferentiated adipocytes. See FIG. 5, Image B, FIG. 6, Images A-B, FIG. 7, Images A-B, FIG. 8, Image B, FIG. 9, Image B, and FIG. 10, Image F. This result is surprising because studies parallel to those of the present disclosure have found that overexpressing these genes in primary cells results in proliferation inhibition. Additionally, previous literature has shown that knocking out MyoD helps transdifferentiate myoblasts into adipocytes. (https://www.sciencedirect.com/science/article/pii/S2352396417300191). Where primary cells have a PDL (population doubling level) limit and a finite lifespan (after which they senesce), the immortal cells of the present invention do not. One possible explanation is that the immortal cells are stuck in a particular part of the cell cycle or a TERT gene is causing the immortal cells to respond differently to this overexpression than primary cells. Another possible explanation is that as the transdifferentiated myoblast cells age and go through many PDLs, they develop higher expression of adipogenesis-related genes, such that even when MyoD is overexpressed it is unable to inhibit the formation of lipid droplets.

Moreover, transdifferentiation of myoblasts to adipocytes has previously been associated with downregulation of MyoD. (Chen et al., Methods Mol Biol., 1889: 25-41 (2019)). The inventors surprisingly discovered the opposite: upregulation of MyoD facilitates myoblast-to-adipocyte transdifferentiation. See FIG. 9, Image B, and FIG. 10, Image F.

Transdifferentiation of fibroblasts to adipocytes has typically been associated with media components in culture, such as hormones and small molecules. These hormones and small molecules are very easy to implement, e.g, by simply adding them to the cell culture media. However, such an approach is not suitable for a consumable product, because commonly used hormones and small molecules are not approved for consumption. On the other hand, genetic engineering approaches tend to result in cells that eventually lose both proliferation capacity and phenotypic characteristics. The inventors surprisingly found a genetic engineering approach that results in cells that retain both transdifferentiated phenotypes and proliferative capacity, such as, for example, a genetic edit that results in overexpression of C/EBPalpha without the use of small molecules and hormones that are not generally recognized as acceptable for consumption. This novel approach to transdifferentiation will facilitate development of consumable products.

Particularly preferred embodiments of this invention include an in-vitro cultured meat product, comprising a population of cells initially comprising fibroblasts, myoblasts, or some combination thereof, wherein the population of cells is transdifferentiated to express adipocyte phenotypes and transfected to induce steatosis via an overexpression of CEPBalpha, CEPBgamma, PPARgamma, SREBP1, SREBP2 or some combination thereof. In certain embodiments, the population of cells may be transfected to downregulate at least one of OSR1, PRRX1, LHX9, TWIST2, and INSIG2. In some embodiments, the transdifferentiation may occur without endogenous hormones or small molecules recognized to transdifferentiate cells into adipocyte phenotypes. In further embodiments, the transdifferentiated population of cells may retain proliferative capacity and exhibit stable phenotype at late passage. In certain embodiments, the population of cells may be transfected to overexpress HNF4alpha and/or to express hepatocyte phenotypes. In some embodiments, the population of cells may include myoblasts having wildtype MyoD. In particular, the wildtype MyoD may be overexpressed. In preferred embodiments, the transdifferentiated population of cells may exhibit lipid droplet formation in the cytoplasm.

In preferred alternative embodiments, the in-vitro cultured meat product may comprise 50-95% in-vitro cultured meat by weight and 5-19% butter, cream, or some combination thereof, by weight. In particular, the in-vitro cultured meat may comprise a population of cells transdifferentiated to express adipocyte phenotypes, a population of cells transdifferentiated to express at least one of hepatocyte phenotypes, adipocyte lineage cells, hepatocyte lineage cells, or some combination thereof. In some embodiments, the butter and/or cream may be combined with or replaced by a plant-based lipid alternative, such as natural oil, canola, vegetable oil, safflower oil, margarine, or some combination thereof. In certain embodiments, the in-vitro cultured meat product may comprise one or more of radishes and carrots at 0.1% to 1% by weight; shallots, garlic, and thyme at 0.5% to 6% by weight; and 1-12% port wine by weight. In certain embodiments, the port wine may be reduced. In some embodiments, the population of cells may be transfected to overexpress at least one of HNF4alpha, liver lineage cells, or some combination thereof. In other embodiments, the in-vitro cultured meat may comprise a population of cells transfected to induce steatosis via an overexpression of CEPBalpha, CEPBgamma, or some combination thereof.

Other preferred embodiments of this invention include a method of cooking an in-vitro cultured meat product, comprising melting a lipid in a cooking apparatus; adding in-vitro cultured meat to the cooking apparatus, wherein the in-vitro cultured meat comprises a population of cells transdifferentiated to express adipocyte phenotypes; and cooking at least one side of the in-vitro cultured meat product until a suitable color change or consistency change is observed, e.g. browned or crisped. In further embodiments, the in-vitro cultured meat may comprise a population of cells transfected to induce steatosis via an overexpression of CEPBalpha, CEPBgamma, or some combination thereof. In certain embodiments, the method may further comprise adding one or more of shallots, garlic, thyme, port wine, salt, and pepper to the in-vitro cultured meat product; blending the in-vitro cultured meat product until smooth with the lipid; and cooling the in-vitro cultured meat product until chilled. In some embodiments, the lipid may comprise plant-based alternatives, such as natural oil, canola, vegetable oil, safflower oil, margarine, or some combination thereof.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

All of the claims in the claim listing are herein incorporated by reference into the specification in their entireties as additional embodiments.

### EXAMPLES

### Example 1: Transdifferentiation of Chicken Embryonic Fibroblasts into Adipocytes by Overexpression of C/EBPalpha

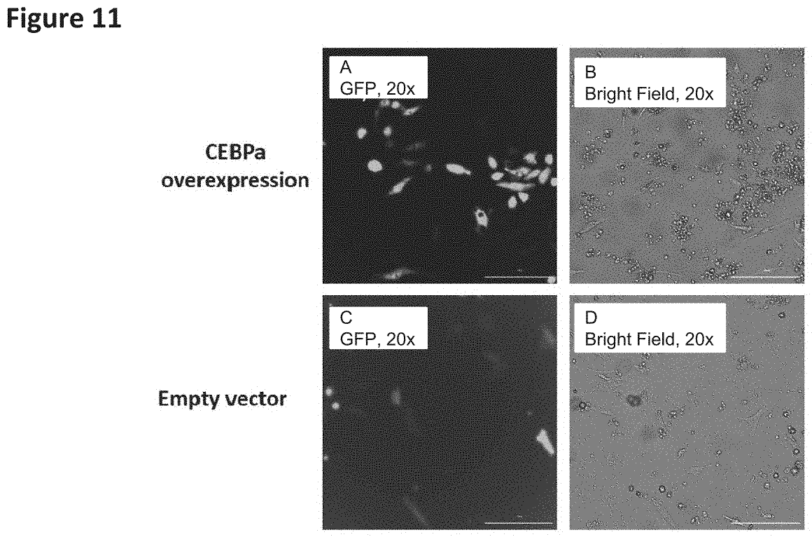

As illustrated by FIG. 11, Images A-B, FIG. 5, Image B, FIG. 6, Images A-B, 2.5×10⁵ chicken embryonic fibroblasts were transfected with a mPGK-ggCEPBa-hEF1a-copGFP-IRES-PuroR vector and PhiC31 integrase expression plasmid in order to transfect the gene C/EBPalpha into fibroblasts to induce transdifferentiation into adipocytes. As illustrated by FIG. 11, Images C-D, FIG. 5, Image A, FIG. 6, Image C, and FIG. 6, Image D, a control population of fibroblasts was transfected with a control, empty vector mPGK-MCS-hEF1a-copGFP-IRES-PuroR. The control population provides a benchmark from which to assess and quantify the efficacy of the vector of interest and its genes. Transfected cells were selected in cultures comprising DMEM-F12 media with 10% FBS, 2% chicken serum, and 100 μg/mL FGF2. In alternative embodiments, cells are grown in serum free media or animal component free (ACF) media. Cells were maintained in puromycin-containing media until non-transfected control cells lost viability (˜72 hours post-transfection). All remaining, viable cells overexpressed the C/EBPalpha gene and exhibited fluorescence from GFP. Selected cells transfected with C/EBPalpha were then grown in cultures comprising DMEM-F12 with 10% FBS, 2% chicken serum, and 100 μg/mL FGF₂ to induce transdifferentiation to adipocytes. In alternative embodiments, cells are grown in serum free media or animal component free (ACF) media. In some instances, cell culture media includes an additional fatty acid supplement. A supplement of fatty acids in the media may result in cells that exhibit an increased fatty acid concentration. The supplemented fatty acids may be taken up the cell and metabolized, may be incorporated into fat globules, or may simply adhere or otherwise get stuck to the cells. Fatty acid supplements for cell culture media may include plant-based lipids.

Cells overexpressing the C/EBPalpha gene began to exhibit formation of lipid droplets in the cytoplasm 72-96 hours post-transfection. Droplets started as little globules, characteristic of committed adipocytes. As cells continued to differentiate, the lipid droplets increased in size as differentiated adipocytes reached maturity. Cell morphology also changed from a fibroblast bipolar or multipolar shape to a mixture of hypertrophic and hyperplastic adipocytes. Fibroblasts were fixed with 10% formalin and stained with Oil Red O in 60% isopropanol and hematoxylin to assess the presence of lipid droplets. Lipid droplets stained red with Oil Red O, and cell nuclei-stained purple with hematoxylin. Lipid droplets stained with Oil Red O also exhibited fluorescence under a Texas Red channel when nuclei were stained with DAPI at a 1:800 ratio.

As shown by comparing FIG. 11, Images A and C, cells transfected to overexpress C/EBPalpha exhibited a clear increase in green fluorescent protein (“GFP”) (FIG. 11, Images A and B) relative to control cells transfected with a control vector (FIG. 11, Images C and D). This result verifies that the vector containing C/EBPalpha was successfully transfected, meaning it traversed the cell membrane, entered the cytoplasm, and began transcription. Where FIG. 11, Images A and C, verify the transcription of GFP by the transfected vector, FIG. 11, Images B and D, verify transcription of C/EBPalpha. Overexpression of C/EBPalpha is characterized by an accumulation of lipids and is a hallmark phenotype of adipocytes. FIG. 11, Image B's bright field image shows a clear increase in lipid droplet formation visible as an increased number of white dots. This series of figures provides proof of transfection success, C/EBPalpha overexpression, and a transition to an adipocyte phenotype.

FIG. 5, Images A-B, FIG. 6, Images A-D, and FIG. 7, Images A-D, provide a series of microscopic images that allow the comparison of unaltered fibroblast morphology relative to the morphology of fibroblasts that have been transdifferentiated to overexpress C/EBPalpha. As shown in FIG. 5, Image A, FIG. 6, Images C-D, and FIG. 7, Images C-D, cells transfected with a control vector retained a fibroblast multipolar morphology showing elongated, lean fibers and failed to exhibit lipid droplets, prior to losing viability. Of particular note, FIG. 5, Image A, fails to show any accumulation of lipids, which would be characterized by groups of white dots. Further controls illustrated by FIG. 6, Images C and D, depict the addition of Oil Red O to the cells samples, which stain lipids with a high visibility red color. However, FIG. 6, Images C and D, fail to show any accumulation of Oil Red O stain, which indicates a lack of lipid accumulation in these controls. Further, FIG. 7, Images A-D, stain the samples with both Oil Red O and DAPI, whereby DAPI stains nuclei of cells with a high visibility blue fluorescence. Although FIG. 7, Image C and FIG. 7, Image D, clearly depict the presence of a multitude of cells with blue fluorescing nuclei, none of these cells fluoresced any Oil Red O, indicating that cells in these controls lacked lipid accumulation. In contrast, as shown in FIG. 5, Image B, FIG. 6, Images A-B, and FIG. 7, Images AB, cells overexpressing C/EBPalpha exhibited transdifferentiation to a new morphology characteristic of adipocytes and a clear increase in the presence of cytoplasmic lipid droplets. Specifically, FIG. 5, Image B, illustrates extensive lipid drop formation as groupings of white dots, clearly in excess to the control, and a reduction of elongated fibroblast multipolar morphology structures relative to the control. FIG. 6, Images A and B, illustrate extensive staining by Oil Red O, which stains lipids, clearly in excess of the control. Finally, FIG. 7, Images A and C, illustrate extensive fluorescence by both Oil Red O and DAPI, indicating the presence of cells having lipid accumulation clearly in excess of the control. Of particular note, cells overexpressing C/EBPalpha exhibited lipid droplet staining as late as 7 days post-transfection.

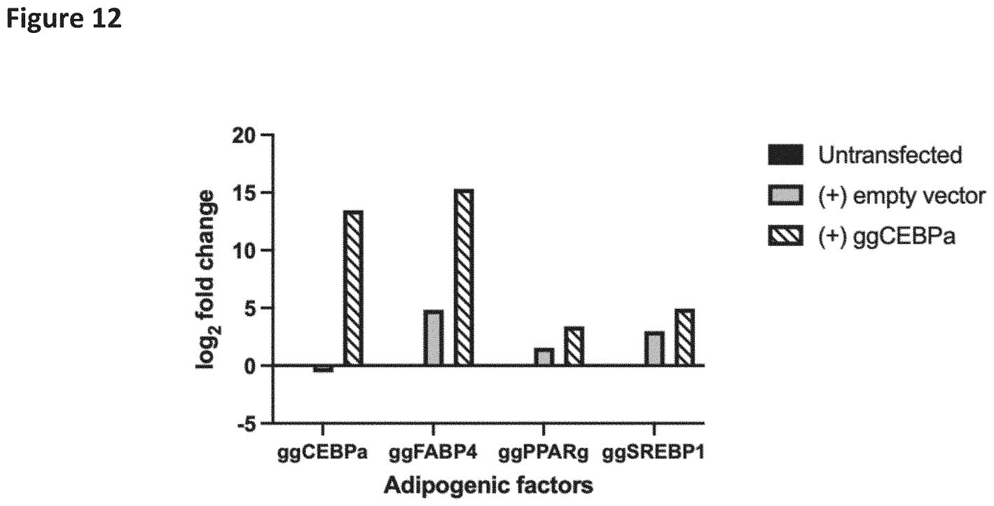

As shown in FIG. 12, cells overexpressing C/EBPalpha exhibited increased mRNA expression of other adipogenic markers such FABP4, PPARgamma, and SREBP1 relative to non-transfected cells or cells transfected with a control, empty vector. This provides further evidence that transfecting fibroblast to overexpress C/EBPalpha results in the transdifferentiation of the fibroblast into adipocytes. Not only do the transdifferentiated cells express the transfected gene C/EBPalpha, but they also express other genes in the adipocyte and lipid generation pathway (i.e. adipogenic factors) in greater abundance than the control and at expression levels characteristic of adipocyte cells. Transfection of vectors designed to overexpress these other, additional adipogenic factors may provide means by which to further increase lipid accumulation, which may be beneficial for foie gras and pate food product formulation. Gene expression was analyzed by qPCR in cells harvested 6 days post-differentiation.

### Example 2: Transdifferentiation of Chicken Embryonic Fibroblasts or Myoblasts into Adipocytes

Chicken embryonic fibroblasts can be derived from day 12 and day 14 chick embryos and grown in DMEM-F12 media with 10% FBS, 2% chicken serum, and 100 μg/mL FGF₂. In alternative embodiments, cells are grown in serum free media or animal component free (ACF) media. Primary myoblasts can be procured from chickens and immortalized.

Genes of interest for overexpression studies, such as PPARgamma, SREBP1, and MyoD can be cloned into a PhiC31 vector. Genes of interest for downregulation studies, such as MyoD1, OSR1, PRRX1, LHX9, TWIST2, and INSIG2, can be downregulated by siRNA and CRISPR guide RNAs. Amaxa™ 4D-Nucleofactor™ can be employed for delivering plasmid DNA into cells at a density of 5×10⁵ cells/reaction.

Phenotypic change can be evaluated by fluorescence microscopy, and lipid droplet formation can be visualized by stain with Oil Red O or BODIPY 493/503. Gene expression analysis can be performed by qPCR.

FIG. 2, Images A-D, provide microscopic images of fibroblast cells transfected to overexpress PPARg in various media conditions. FIG. 2, Images A and B, illustrate limited lipid accumulation when the cells overexpressing PPARg are grown in fibroblast media both lacking oleic acid (FIG. 2, Image A) and having oleic acid (FIG. 2, Image B). FIG. 2, Images C and D, both illustrate increased lipid accumulation when the cells overexpressing PPARg are grown in adipocyte differentiation media both lacking oleic acid (FIG. 2, Image C) and having oleic acid (FIG. 2, Image D). In particular, FIG. 2, Images C and D, clearly show lipid accumulation and a reduction in elongated multipolar morphology, which is characteristic of fibroblast cells. In contrast, FIG. 2, Images A and B, clearly show a lack of lipid accumulation and a retention of the fibroblast's elongated multipolar morphology. FIG. 2, Images A-D, therefore illustrate that transdifferentiation of fibroblast into adipocytes using PPARg overexpression is enhanced by certain media conditions. FIG. 4, Images A-D, depict the same variables explored in FIG. 2, Images A-D, with the addition of red stain for lipids (i.e. Oil Red 0) and dark purple stain for nuclei (i.e. hematoxylin). FIG. 4, Images A and B, illustrate limited lipid accumulation for cells overexpressing PPARg in fibroblast media both lacking oleic acid (FIG. 4, Image A) and having oleic acid (FIG. 4, Image B). This conclusion is supported by the lack of red stain in FIG. 4, Images A and 4B. In contrast, FIG. 4, Images C and D, illustrate robust lipid accumulation for cells overexpressing PPARg in adipocyte differentiation media as indicated by the red staining visible in both figures.

FIG. 3 provides a graph quantifying mRNA expression of adipogenic markers PPARg, CEBPa, FABP, and SREBP1 as analyzed by qPCR in cells harvesed 6 days post-differentiation. The analyzed cells include those having an empty vector and those transfected to overexpress PPARg in either fibroblast media (FM), adipocyte differentiation media (ADM), fibroblast and oleic acid media, and adipocyte differentiation and oleic acid media. These results indicate that overexpression of PPARg alone is insufficient to upregulate all tested adipogenic markers. Instead, upregulation of these adipogenic markers requires a combination of overexpression of PPARg and proper media conditions. This experiment indicates ADM media provides the best conditions for upregulating adipogenic markers in cells transfected to overexpress PPARg, while the addition of oleic acid further increases the upregulation of these adipogenic markers.

FIG. 8, Images A and B, transition to a comparison of fibroblast cells transfected with an empty vector (FIG. 10, Image A) and TERT-immortalized chicken embryonic fibroblasts overexpressing CEBPa (FIG. 10, Image B), as previously examined in FIG. 5, Images A-B, FIG. 6, Images A-D, FIG. 7, Images A-D, FIG. 11, Images A-D, and FIG. 12. FIG. 8, Image A, illustrates that fibroblast cells transfected with an empty vector do not exhibit the presence of lipid droplets or lipid accumulation. In contrast, FIG. 8, Image B, illustrates that TERT-immortalized chicken embryonic fibroblasts overexpressing CEBPa accumulate lipids in their cytoplasms and transition away from a fibroblast morphology, which indicates their transdifferentiation into adipocytes. Surprisingly, these transdifferentiated cells also maintained their proliferative capacity as evidenced by their continued expansion and proliferation, even when simultaneously accumulating lipid droplets. Typically, cells lose proliferation capacity upon differentiation.

FIG. 9, Images A and B, transition to a comparison of chicken embryonic myoblast cells overexpressing MyoD in either myoblast media (FIG. 9, Image A) or adipocyte differentiation media (FIG. 9, Image B). As shown by FIG. 9, Image A, MyoD-expressing cells do not differentiate in myoblast media even upon addition of 500 uM of oleic acid. In contrast, as shown by FIG. 9, Image B, switching the media to adipocyte differentiation media and adding 500 uM of oleic acid result in the formation of lipid droplets.

FIG. 10, Images A-F, compare six different conditions for lipid accumulation in immortalized chicken embryonic myoblast. The myoblast either lack MyoD overexpression (FIG. 10, Images A-C) or have MyoD overexpression (FIG. 10, Images D-F). Lipid accumulation for these two cell type variants is tested against three different media conditions: myoblast media (FIG. 10, Images A and D), myoblast media supplemented with oleic acid (FIG. 10, Images B and E), and adipocyte differentiation media supplemented with oleic acid (FIG. 10, Images C and F). FIG. 10, Images A and D, illustrate that myoblasts cultured in myoblast growth media, with (FIG. 10, Image D) and without (FIG. 10, Image A) MyoD overexpression, retained myoblast morphology and failed to transdifferentiate into adipocytes. Similarly, FIG. 10, Image B and E, illustrate that myoblasts cultured in myoblast growth media in the presence of 500 μM of oleic acid, with (FIG. 10, Image E) and without (FIG. 10, Image B) MyoD overexpression, retained myoblast morphology and failed to transdifferentiate into adipocytes. In contrast, FIG. 10, Images C and F, illustrate that myoblasts cultured in adipocyte differentiation media in the presence of 500 μM of oleic acid, with (FIG. 10, Image C) and without (FIG. 10, Image F) MyoD overexpression, successfully transdifferentiate into adipocytes, thus demonstrating the criticality of adipocyte differentiation media to myoblast transdifferentiation. Transdifferentiation, however, was more robust in cells that overexpressed MyoD (FIG. 10, Image F), demonstrating that MyoD overexpression also plays a role in the degree of transdifferentiation.

### Example 3: Generation of Hepatocytes Exhibiting Steatosis

Primary hepatocytes can be procured from ducks, geese, or chickens and expanded and immortalized.

Genes of interest for overexpression studies, such as PPARgamma, C/EBPalpha, SREBP1, and SREBP2, can be cloned into a PhiC31 vector. Genes of interest for downregulation studies, such as OSR1, PRRX1, LHX9, TWIST2, and INSIG2, can be downregulated by siRNA and CRISPR guide RNAs. Amaxa™ 4D-Nucleofactor™ can be employed for delivering plasmid DNA into cells at a density of 5×10⁵ cells/reaction.

Phenotypic change can be evaluated by fluorescence microscopy, and lipid droplet formation can be visualized by stain with Oil Red O or BODIPY 493/503. Gene expression analysis can be performed by qPCR.

### Example 4: Transdifferentiation of Fibroblasts into Hepatocytes

Primary fibroblasts can be procured from ducks and chickens and immortalized. Cells can be grown in DMEM-F12 media with 10% FBS, 2% chicken serum, and 100 μg/mL FGF₂. In alternative embodiments, cells are grown in serum free media or animal component free (ACF) media.

Genes of interest for overexpression studies can be cloned into a PhiC31 vector. Such genes include: ATF5, PROX1, FOXA2, FOXA3, HNF4A, ONECUT1, NR1H4, MLXIPL, NR5A2, and XBP1. Amaxa™ 4D-Nucleofactor™ can be employed for delivering plasmid DNA into cells at a density of 5×10⁵ cells/reaction.

Phenotypic change can be evaluated by fluorescence microscopy, and lipid droplet formation can be visualized by stain with Oil Red O or BODIPY 493/503. Gene expression analysis can be performed by qPCR.

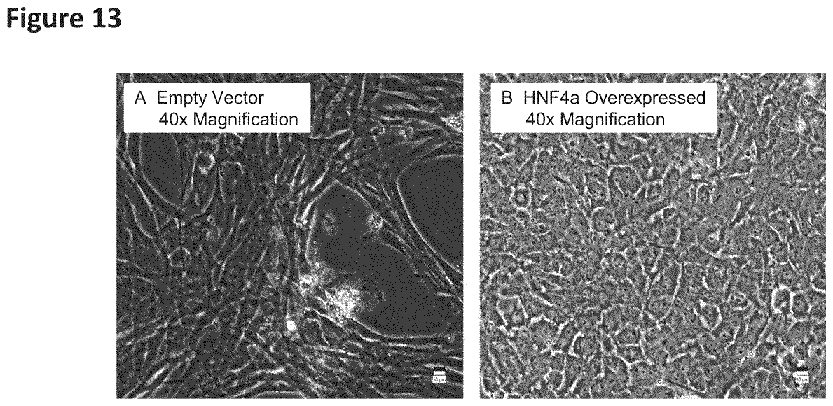

FIG. 13, Images A and B, illustrate chicken embryonic fibroblast transfected with an empty vector (FIG. 13, Image A) and transfection with a vector causing overexpression of HNF4a (FIG. 13, Image B). FIG. 13, Image A, shows that the chicken embryonic fibroblast retained fibroblast morphology when transfected with an empty vector. In contrast, FIG. 13, Image B, shows that chicken embryonic fibroblast transfected to overexpress HNF4a transdifferentiated to exhibit a hepatocyte morphology 10 days post-transfection.

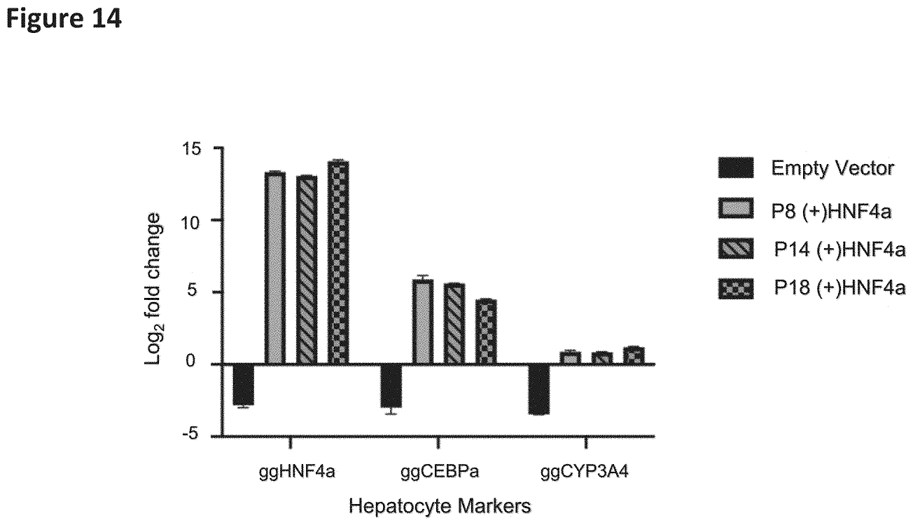

FIG. 14 provides a graph quantifying mRNA expression of hepatocyte markers HNF4a, CEBPa, and CYP3A4 as analyzed by qPCR in immortalized chicken embryonic fibroblast cells harvested 6 days post-differentiation. The analyzed cells include those having an empty vector and those transfected to overexpress HNF4a at a passage level (i.e. number of population doublings) of either 8, 14, or 18. These results indicate that immortalized chicken embryonic fibroblast cells transfected to overexpress HNF4a exhibit phenotype stability even as they proliferate. Much like the results of FIG. 8, Image B, the transdifferentiated cells of FIG. 14 surprisingly maintained their proliferative capacity along with a stable transdifferentiated phenotype across extensive population doublings.

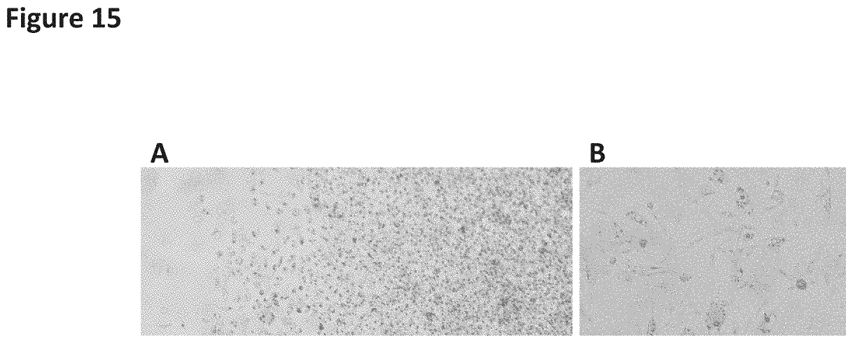

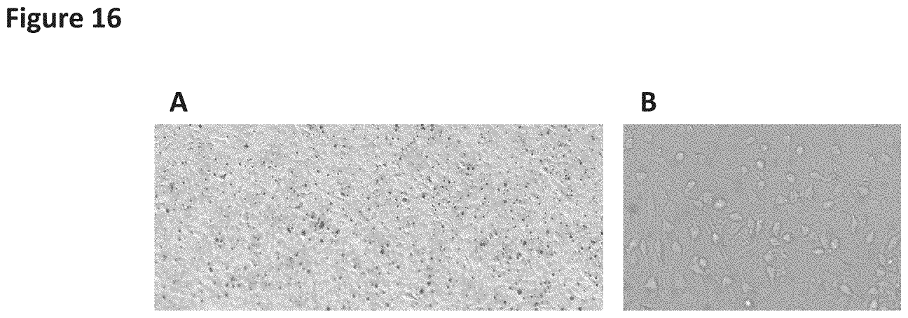

As shown in FIG. 15, Images A-B, and FIG. 16, Images A-B, lipid accumulation also occurs in cells during larger scale production runs, as, in separate experiments, chicken fibroblasts overexpressing either C/EBPalpha or HNF4alpha exhibited such accumulation while being grown in BR7 bioreactors. Verification that benchtop results translate to larger scale growing methods is of paramount concern when aiming to mass produce cell-based meat for consumption. As cell growing techniques are scaled from the benchtop to large applications, it is common to see cell phenotypes change or disappear over time. Similarly, native myoblasts typically have the capacity to differentiate when they are young, but they often lose this ability as they age. The reason that cells lose their ability to proliferate and lose their ability to differentiate is poorly understood but is suspected to be related to an aging process and/or epigenetic in origin. As such, it is difficult to predict whether benchtop techniques for cell cultivation will scale up successfully. Of particular note, FIG. 15, Images A and B, illustrate that fibroblast cells transfected to overexpress CEBPa and grown in BR7 roller bottle bioreactors exhibit a high lipid accumulation at 60-80% confluence. Similarly, FIG. 16, Images A and B, illustrate that HNF4alpha-overexpressing fibroblasts exhibited cuboidal morphology characteristic of hepatocytes and therefore indicative of successful transdifferentiation. As such, both FIG. 15, Images A-B, and FIG. 16, Images A-B, show that TERT immortalized chicken embryonic fibroblasts transfected to overexpress both CEBPa and HNF4a exhibited phenotype stability of adipocytes and hepatocytes, respectively, along with the retention of proliferative capacity, even when the cells were grown at large scale in a bioreactor, therefore proving the efficacy of the disclosed methods for mass producing cell based meat.

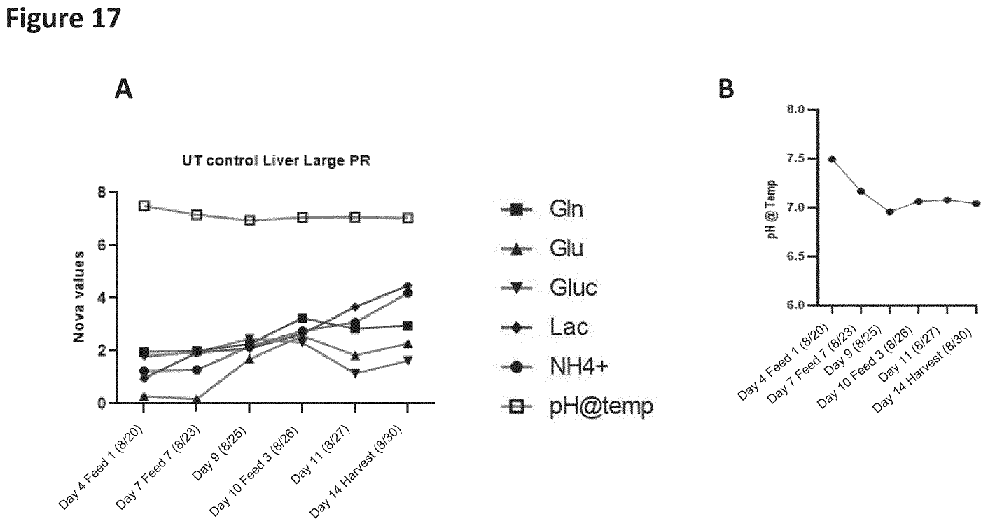

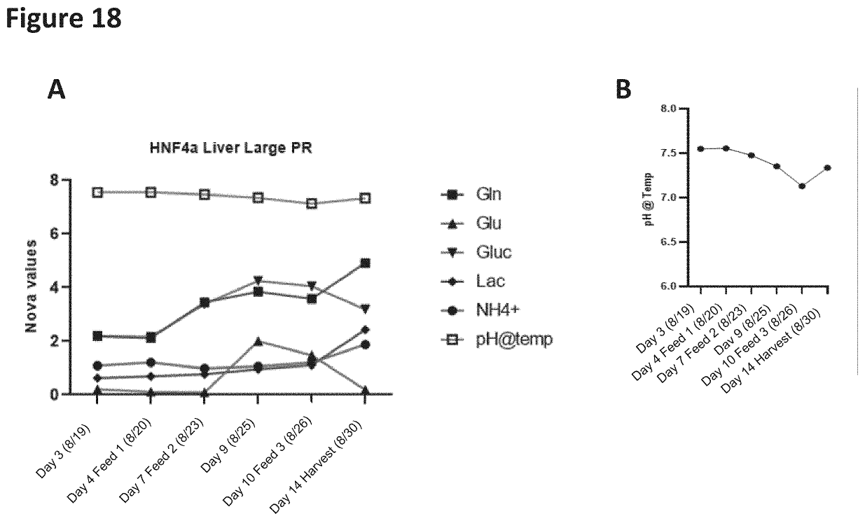

FIG. 17, Graphs A-B, and FIG. 18, Graphs A-B, illustrate the variation of metabolites, waste products, and pH of cell-based meat cultivated during a large-scale production run lasting 14 days. FIG. 17, Graphs A and B, illustrate the growth of chicken fibroblast cells, which serves as a control in this case, as they grow over 14 days in a large-scale production run. The buildup of lactate and ammonia and the acidification of the media are indicative of active cellular metabolism and active proliferation and may serve as proxies for rate of growth. FIG. 18, Graphs A and B, illustrate the growth of fibroblasts transdifferentiated into hepatocytes, by overexpression of HNF4a, as they grow over 14 days in a large-scale production run. Again, the buildup of lactate and ammonia and the acidification of the media are indicative of active cellular metabolism and active proliferation and may serve as proxies for rate of growth. However, of particular note, ammonia built up to a lesser degree in FIG. 18, Graph A, as compared to FIG. 17, Graph A. This may be advantageous during the early stages of cell growth because excessive ammonia buildup may compromise tissue quality and may result in premature detachment of tissue from its growing substrate. On the other hand, allowing ammonia to build up in anticipation of harvest may ease the collection of the cells as the ammonia build may facilitate cell detachment from the growth substrate. In addition to metabolite analysis, the cell-based meat is also analyzed to determine the quality of tissue formed. In some embodiments, cell-based meat is grown in an adherent culture and forms a thin sheet of cells once the growth reaches confluence. Once a tissue sheet is grown and collected, tissue quality is evaluated according to a 5-point scale. A score of 1 would be attributed to a product that cannot retain a sheet structure when manipulated because it rips and shreds even when lightly touched. A familiar example that would score a 1 on the tissue quality scale would be a sheet of ground parmesan cheese, which falls apart immediately when handled. On the other hand, a score of 5 would be attributed to a product that forms an intact sheet of cells that does not rip even when held in one's hands and pulled on. A familiar example that may score a 5 in certain situations is a well-formed pizza dough. Turning to the results of the present disclosure, the non-transfected, control cells of FIG. 17, Graphs A and B, obtained a tissue quality score of 5/5, while HNF4alpha-overexpressing chicken fibroblasts of FIG. 18, Graphs A and B, obtained a tissue quality score of 4.5/5. Similarly, C/EBPalpha-overexpressing chicken fibroblasts also maintained normal function and properties during larger-scale production runs and exhibited a tissue quality score of 5/5.

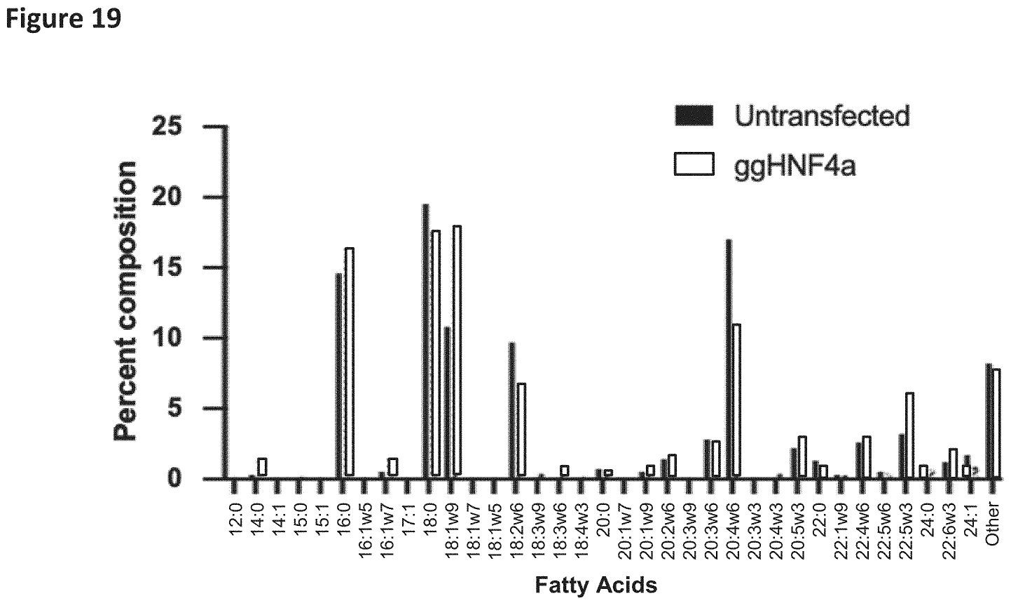

FIG. 19 quantifies the fatty acid profile of embryonic chicken fibroblast transdifferentiated into hepatocytes by overexpression of HFN4a as compared to a control fibroblast tissue. Liver products, such as foie gras, are not only high in fatty acid content but also high in specific types of fatty acids. The results shown by FIG. 19 show that embryonic chicken fibroblast transdifferentiated into hepatocytes by overexpression of HFN4a exhibit a fatty acid profile similar to foie gras. Omega-3 fatty acids, such as oleic acid, alpha-linolenic acid, eicosapentaenoic acid, docosapentaenoic acid, and docosahexaenoic acid, are abundantly present in conventional foie gras and similarly high expression is shown by FIG. 19 for embryonic chicken fibroblast transdifferentiated into hepatocytes by overexpression of HFN4a. Of particular note, the control did not achieve the same high levels of omega-3 fatty acids.

The following Table 2 provides a quantitative analysis of the fatty acid profile of cells overexpressing CEBPs. Samples F1-F3 comprise untransfected controls, while samples F4-F7 comprise cells transfected to overexpress CEBPa. In these examples, overexpression of CEBPa led to an increase in relative concentration of both palmitic acid and palmitoleic acid. Both are saturated fats that enable the formation of well-formed fat globules. Additionally, these fatty acids are the predominant fatty acids present in adipocyte cells. This quantitative analysis therefore supports the conclusion that CEBPa transfection facilitates the transdifferentiation of fibroblast cells into adipocyte cells.

TABLE 2
Percent Composition (Area Percent all fatty acids total 100%)

| | | AAC | | | | | | |
|---|---|---|---|---|---|---|---|---|
| | | 58094 | 58095 | 58096 | 58097 | 58098 | 58099 | 58100 |
| | Sample | F1 | F2 | F3 | F4 | F5 | F6 | F7 |
| Lauric acid | 12:0 | 0 | 0 | 0 | 0 | 0 | 0 | 0 |
| Myristic | 14:0 | 1.3 | 1 | 1.1 | 1.6 | 1.4 | 1.4 | 1.5 |
| Myristoleic | 14:1 | 0 | 0 | 0 | 0 | 0 | 0 | 0 |
| Pentadecanoic | 15:0 | 0.2 | 0.2 | 0.2 | 0.2 | 0.2 | 0.2 | 0.2 |
| | 15:1 | 0 | 0.6 | 0.5 | 0.1 | 0.1 | 0.1 | 0.1 |
| Palmitic | 16:0 | 14.9 | 15.1 | 15.3 | 18.2 | 17.9 | 16.6 | 16.8 |
| | 16:1w5 | 0 | 0 | 0 | 0 | 0 | 0 | 0 |
| Palmitoleic | 16:1w7 | 1.3 | 1.2 | 1.2 | 1.8 | 1.6 | 1.6 | 1.7 |
| | 17:1 | 0.4 | 1.7 | 1.3 | 0.1 | 0.1 | 0.4 | 0.4 |
| Stearic | 18:0 | 19.9 | 19.9 | 20 | 19.2 | 18.9 | 18.7 | 18.8 |
| Oleic | 18:1w9 | 18.5 | 18.8 | 18.9 | 18.3 | 18.5 | 19 | 19 |
| Vaccenic | 18:1w7 | 0 | 0 | 0 | 0 | 0 | 0 | 0 |
| | 18:1w5 | 0 | 0 | 0 | 0 | 0 | 0 | 0 |
| Linoleic | 18:2w6 | 8.8 | 9.2 | 9.1 | 7.2 | 7.6 | 7.8 | 7.8 |
| gamma-linolenic | 18:3w6 | 0.1 | 0.1 | 0.2 | 0.1 | 0.1 | 0.1 | 0.1 |
| alpha-linolenic | 18:3w3 | 0.4 | 0.6 | 0.6 | 0 | 0.4 | 0.3 | 0.4 |
| | 18:4w3 | 0.2 | 0.2 | 0.2 | 0.3 | 0.2 | 0.2 | 0.2 |
| Arachidic | 20:0 | 0.4 | 0.4 | 0.4 | 0.4 | 0.4 | 0.4 | 0.4 |
| | 20:1w7 | 0 | 0 | 0 | 0 | 0 | 0 | 0 |
| 11-Eicoenoic | 20:1w9 | 0.4 | 0.5 | 0.5 | 0.4 | 0.5 | 0.6 | 0.5 |
| Eicosadienoic | 20:2w6 | 0.8 | 0.9 | 0.9 | 0.9 | 0.9 | 0.9 | 0.8 |
| Mead's acid | 20:3w9 | 0 | 0 | 0 | 0 | 0 | 0 | 0 |
| Dihomogamma- | 20:3w6 | 2.6 | 2.7 | 2.7 | 2.5 | 2.4 | 2.4 | 2.4 |
| linolenic acid | | | | | | | | |
| Arachidonic | 20:4w6 | 8.8 | 7.7 | 7.9 | 8.7 | 8.7 | 8.4 | 8.2 |
| | 20.3w3 | 0.1 | 0.1 | 0.1 | 0.1 | 0.1 | 0.1 | 0.1 |
| | 20:4w3 | 0.2 | 0.4 | 0.4 | 0.2 | 0.2 | 0.2 | 0.2 |
| Eicosapentaenoic | 20:5w3 | 2.4 | 2.2 | 2.1 | 2.5 | 2.3 | 2.4 | 2.4 |
| Behenic | 22:0 | 0.7 | 0.8 | 0.8 | 0.7 | 0.7 | 0.7 | 0.7 |
| Erucic | 22:1w9 | 0.1 | 0.3 | 0.4 | 0.2 | 0.2 | 0.4 | 0.4 |
| Docosatetraenoic | 22:4w6 | 2.7 | 2.3 | 2.4 | 2.3 | 2.3 | 2.3 | 2.2 |
| Docosapentaenoic- | 22:5w6 | 0.3 | 0.4 | 0.4 | 0.3 | 0.3 | 0.4 | 0.4 |
| omega 6 | | | | | | | | |
| Docosapentaenocic- | 22:5w3 | 4.2 | 4.6 | 4.4 | 4 | 3.9 | 4.3 | 4.2 |
| omega 3 | | | | | | | | |
| Lignoceric | 24:0 | 0.4 | 0.4 | 0.5 | 0.6 | 0.5 | 0.5 | 0.4 |
| Docosahexaenoic | 22:6w3 | 1.7 | 1.6 | 1.5 | 1.7 | 1.7 | 1.7 | 1.7 |
| Nervonic | 24:1 | 0.9 | 0.9 | 0.9 | 1 | 0.9 | 0.9 | 0.9 |
| | other | 7.2 | 5.2 | 5.1 | 6.5 | 7.2 | 7.1 | 7.4 |
| | sum | 100 | 100 | 100 | 100 | 100 | 100 | 100 |
| | Saturates | 37.9 | 37.9 | 38.3 | 40.8 | 40.1 | 38.5 | 38.8 |
| | Monoenes | 21.5 | 24 | 23.7 | 21.9 | 21.9 | 22.8 | 22.9 |
| | PUFA | 33.3 | 32.9 | 32.9 | 30.8 | 30.9 | 31.6 | 30.9 |
| | HUFA | 23.7 | 22.8 | 22.9 | 22.7 | 22.5 | 22.9 | 22.4 |
| | T/T | 0 | 0 | 0 | 0 | 0 | 0 | 0 |
| | Tot. w3 | 9.2 | 9.6 | 9.3 | 8.7 | 8.7 | 9.2 | 9.1 |
| | Tot. w6 | 24.1 | 23.2 | 23.6 | 22 | 22.2 | 22.4 | 21.9 |
| | Tot. w9 | 19.9 | 20.5 | 20.7 | 19.9 | 20.1 | 20.8 | 20.8 |
| | w6/w3 | 2.6 | 2.4 | 2.5 | 2.5 | 2.5 | 2.4 | 2.4 |
| | 20:4/20:5 | 3.7 | 3.6 | 3.8 | 3.6 | 3.7 | 3.5 | 3.5 |

The following Table 3 provides a quantitative analysis of the fatty acid profile of cells overexpressing HNF4a. Samples F1-F4 comprise untransfected controls, while samples F5-F8 comprise cells transfected to overexpress HNF4a. In these examples, overexpression of HNF4a led to an increased concentration of palmitic acid, palmitoleic acid, stearic acid, oleic acid, linoleic acid, and many others as captured below, as expressed in ug/g. Of particular note, linoleic acid is the highest percentage fat in foie gras. This quantitative analysis therefore supports the conclusion that HNF4a transfection supports the transdifferentiation of fibroblast cells into liver cells having fatty acid profiles conducive to the formation of a foie gras food product.

TABLE 3
Tissue Total Lipid Fatty Acids: ug/g

| | | AAC | | | | | | | |
|---|---|---|---|---|---|---|---|---|---|
| | | 58515 | 58516 | 58517 | 58518 | 58519 | 58520 | 58521 | 58522 |
| | | F1 | F2 | F3 | F4 | F5 | F6 | F7 | F8 |
| | Sample | UT1 | UT2 | UT3 | UT4 | HNF4a 1 | HNF4a 2 | HNF4a 3 | HNF4a 4 |
| | Total Fatty | 5596 | 6440 | 5727 | 6252 | 10598 | 9946 | 10165 | 13703 |
| | Acid (ug/g) | | | | | | | | |
| | Percent (%) | 0.59 | 0.68 | 0.60 | 0.66 | 1.12 | 1.05 | 1.07 | 1.44 |
| | of Mass | | | | | | | | |
| Lauric acid | 12:0 | 0.0 | 0.0 | 0.0 | 0.0 | 0.0 | 0.0 | 0.0 | 0.0 |
| Myristic | 14:0 | 80.5 | 83.2 | 109.9 | 109.1 | 145.0 | 123.7 | 135.4 | 169.3 |
| Myristoleic | 14:1 | 0.0 | 0.0 | 0.0 | 0.0 | 0.0 | 0.0 | 0.0 | 0.0 |
| Pentadecanoic | 15:0 | 14.9 | 14.4 | 16.3 | 15.9 | 20.5 | 19.4 | 20.9 | 28.5 |
| | 15:1 | 51.2 | 67.5 | 10.9 | 13.7 | 13.0 | 22.9 | 9.3 | 10.3 |
| Palmitic | 16:0 | 814.7 | 952.4 | 912.7 | 989.6 | 2057.1 | 1899.4 | 1973.4 | 2628.6 |
| | 16:1w9 | 0.0 | 0.0 | 0.0 | 0.0 | 0.0 | 0.0 | 0.0 | 0.0 |
| Palmitoleic | 16:1w7 | 89.9 | 100.8 | 103.1 | 104.3 | 185.6 | 168.1 | 182.4 | 228.8 |
| | 17:1 | 130.1 | 164.0 | 26.3 | 35.6 | 46.0 | 86.6 | 30.2 | 37.0 |
| Stearic | 18:0 | 1060.7 | 1212.6 | 1062.7 | 1170.7 | 1901.4 | 1809.4 | 1809.2 | 2434.0 |
| Oleic | 18:1w9 | 1134.1 | 1313.4 | 1199.4 | 1292.3 | 1947.3 | 1856.8 | 1865.2 | 2443.3 |
| Vaccenic | 18:1w7 | 0.0 | 0.0 | 0.0 | 0.0 | 0.0 | 0.0 | 0.0 | 0.0 |
| | 18:1w5 | 0.0 | 0.0 | 0.0 | 0.0 | 0.0 | 0.0 | 0.0 | 0.0 |
| Linoleic | 18:2w6 | 410.3 | 448.3 | 399.2 | 416.1 | 543.1 | 524.2 | 509.4 | 725.9 |
| gamma- | 18:3w6 | 7.5 | 7.1 | 6.8 | 6.7 | 17.1 | 13.8 | 17.1 | 21.4 |
| linolenic | | | | | | | | | |
| alpha- | 18:3w3 | 20.4 | 25.9 | 17.8 | 22.6 | 28.1 | 26.3 | 27.3 | 36.3 |
| linolenic | | | | | | | | | |
| | 18:4w3 | 14.2 | 19.0 | 12.0 | 14.3 | 13.1 | 17.2 | 10.8 | 16.7 |
| Arachidic | 20:0 | 26.7 | 28.5 | 24.7 | 25.5 | 49.3 | 49.5 | 49.1 | 64.8 |
| | 20:1w7 | 0.0 | 0.0 | 0.0 | 0.0 | 0.0 | 0.0 | 0.0 | 0.0 |
| 11-Eicoenoic | 20:1w9 | 27.9 | 33.1 | 29.9 | 34.7 | 62.8 | 61.0 | 59.0 | 74.9 |
| Eicosadienoic | 20:2w6 | 47.0 | 54.7 | 48.7 | 59.5 | 146.1 | 137.7 | 134.5 | 173.5 |
| Mead's acid | 20:3w9 | 20.9 | 22.5 | 21.1 | 23.8 | 32.7 | 29.5 | 31.3 | 41.6 |
| Dihomogamma- | 20:3w6 | 133.7 | 148.6 | 134.4 | 150.7 | 211.5 | 191.1 | 194.9 | 254.0 |
| linolenic acid | | | | | | | | | |
| Arachidonic | 20:4w6 | 460.2 | 500.3 | 445.6 | 508.8 | 913.3 | 905.1 | 925.3 | 1300.4 |
| | 20.3w3 | 2.8 | 4.0 | 3.1 | 4.1 | 4.0 | 4.0 | 4.0 | 6.0 |
| | 20:4w3 | 11.0 | 14.4 | 10.7 | 13.2 | 12.9 | 12.1 | 11.3 | 16.0 |
| Eicosapentaenoic | 20:5w3 | 141.1 | 170.4 | 139.8 | 158.6 | 238.5 | 232.9 | 219.4 | 301.1 |
| Behenic | 22:0 | 39.0 | 43.5 | 36.8 | 39.5 | 92.3 | 85.2 | 86.9 | 118.2 |
| Erucic | 22:1w9 | 15.6 | 18.2 | 13.7 | 13.2 | 35.8 | 16.5 | 18.3 | 24.8 |
| Docosatetraenoic | 22:4w6 | 132.6 | 149.1 | 140.5 | 153.4 | 202.1 | 197.9 | 210.7 | 289.2 |
| Docosapentaenoic- | 22:5w6 | 2.5 | 3.7 | 19.7 | 22.6 | 35.0 | 5.7 | 5.7 | 13.5 |
| omega 6 | | | | | | | | | |
| Docosapentaenocic- | 22:5w3 | 242.2 | 300.8 | 263.0 | 305.7 | 548.0 | 536.9 | 528.2 | 732.5 |
| omega 3 | | | | | | | | | |
| Lignoceric | 24:0 | 16.4 | 24.8 | 21.2 | 21.7 | 45.3 | 48.0 | 46.6 | 63.0 |
| Docosahexaenoic | 22:6w3 | 92.7 | 115.3 | 93.2 | 114.6 | 212.6 | 204.4 | 208.3 | 281.6 |
| Nervonic | 24:1 | 40.8 | 47.7 | 45.4 | 42.1 | 67.5 | 65.1 | 67.3 | 81.8 |
| | other | 314.1 | 352.5 | 358.2 | 369.0 | 770.7 | 596.1 | 773.6 | 1086.2 |
| | sum | 5596 | 6440 | 5726.8 | 6251.5 | 10598.0 | 9946.4 | 10164.9 | 13703.2 |
| | Total | 2052.8 | 2359.4 | 2184.4 | 2372.0 | 4311.0 | 4034.6 | 4121.6 | 5506.4 |
| | Saturates | | | | | | | | |
| | Total | 1489.6 | 1744.7 | 1428.7 | 1535.8 | 2358.1 | 2276.9 | 2231.7 | 2900.9 |
| | Monounsaturates | | | | | | | | |
| | Total | 1739.1 | 1983.9 | 1755.6 | 1974.8 | 3158.2 | 3038.8 | 3038.1 | 4209.7 |
| | Polyunsaturates | | | | | | | | |
| | HUFA | 1281.8 | 1480.9 | 1307.7 | 1499.1 | 2469.0 | 2376.9 | 2394.2 | 3310.2 |
| | T/T | 0.0 | 0.0 | 0.0 | 0.0 | 0.0 | 0.0 | 0.0 | 0.0 |
| | Total | 524.4 | 649.7 | 539.6 | 633.1 | 1057.2 | 1033.8 | 1009.3 | 1390.1 |
| | Omega 3 | | | | | | | | |
| | Total | 1193.8 | 1311.7 | 1194.9 | 1317.8 | 2068.3 | 1975.5 | 1997.5 | 2778.0 |
| | Omega 6 | | | | | | | | |
| | Total | 1218.5 | 1412.3 | 1288.4 | 1382.3 | 2113.4 | 1999.4 | 2009.8 | 2624.8 |
| | Omega 9 | | | | | | | | |
| | Omega 6/ | 2.3 | 2.0 | 2.2 | 2.1 | 2.0 | 1.9 | 2.0 | 2.0 |
| | Omega 3 | | | | | | | | |
| | Ratio | | | | | | | | |
| | 20:4/20:5 | 3.3 | 2.9 | 3.2 | 3.2 | 3.8 | 3.9 | 4.2 | 4.3 |

Estimate of total lipid based on recovery of odd chain free fatty acid.

The following Table 4 provides a quantitative analysis of the fatty acid profile of cells overexpressing HNF4a. Samples F1-F4 comprise untransfected controls, while samples F5-F8 comprise cells transfected to overexpress HNF4a. In these examples, overexpression of HNF4a led to an increased concentration of palmitic acid, palmitoleic acid, stearic acid, oleic acid, linoleic acid, and many others as captured below, as expressed in relative percent composition.

TABLE 4
Percent Composition (Area Percent all fatty acids total 100%)

| | | AAC | | | | | | | |
|---|---|---|---|---|---|---|---|---|---|
| | | 58515 | 58516 | 58517 | 58518 | 58519 | 58520 | 58521 | 58522 |
| | Sample | F1 | F2 | F3 | F4 | F5 | QF6 | F7 | F8 |
| Lauric acid | 12:0 | 0.0 | 0.0 | 0.0 | 0.0 | 0.0 | 0.0 | 0.0 | 0.0 |
| Myristic | 14:0 | 1.4 | 1.3 | 1.9 | 1.7 | 1.4 | 1.2 | 1.3 | 1.2 |
| Myristoleic | 14:1 | 0.0 | 0.0 | 0.0 | 0.0 | 0.0 | 0.0 | 0.0 | 0.0 |
| Pentadecanoic | 15:0 | 0.3 | 0.2 | 0.3 | 0.3 | 0.2 | 0.2 | 0.2 | 0.2 |
| | 15:1 | 0.9 | 1.0 | 0.2 | 0.2 | 0.1 | 0.2 | 0.1 | 0.1 |
| Palmitic | 16:0 | 14.6 | 14.8 | 15.9 | 15.8 | 19.4 | 19.1 | 19.4 | 19.2 |
| | 16:1w5 | 0.0 | 0.0 | 0.0 | 0.0 | 0.0 | 0.0 | 0.0 | 0.0 |
| Palmitoleic | 16:1w7 | 1.6 | 1.6 | 1.8 | 1.7 | 1.8 | 1.7 | 1.8 | 1.7 |
| | 17:1 | 2.3 | 2.5 | 0.5 | 0.6 | 0.4 | 0.9 | 0.3 | 0.3 |
| Stearic | 18:0 | 19.0 | 18.8 | 18.6 | 18.7 | 17.9 | 18.2 | 17.8 | 17.8 |
| Oleic | 18:1w9 | 20.3 | 20.4 | 20.9 | 20.7 | 18.4 | 18.7 | 18.3 | 17.8 |
| Vaccenic | 18:1w7 | 0.0 | 0.0 | 0.0 | 0.0 | 0.0 | 0.0 | 0.0 | 0.0 |
| | 18:1w5 | 0.0 | 0.0 | 0.0 | 0.0 | 0.0 | 0.0 | 0.0 | 0.0 |
| Linoleic | 18:2w6 | 7.3 | 7.0 | 7.0 | 6.7 | 5.1 | 5.3 | 5.0 | 5.3 |
| gamma-linolenic | 18:3w6 | 0.1 | 0.1 | 0.1 | 0.1 | 0.2 | 0.1 | 0.2 | 0.2 |
| alpha-linolenic | 18:3w3 | 0.4 | 0.4 | 0.3 | 0.4 | 0.3 | 0.3 | 0.3 | 0.3 |
| | 18:4w3 | 0.3 | 0.3 | 0.2 | 0.2 | 0.1 | 0.2 | 0.1 | 0.1 |
| Arachidic | 20:0 | 0.5 | 0.4 | 0.4 | 0.4 | 0.5 | 0.5 | 0.5 | 0.5 |
| | 20:1w7 | 0.0 | 0.0 | 0.0 | 0.0 | 0.0 | 0.0 | 0.0 | 0.0 |
| 11-Eicoenoic | 20:1w9 | 0.5 | 0.5 | 0.5 | 0.6 | 0.6 | 0.6 | 0.6 | 0.5 |
| Eicosadienoic | 20:2w6 | 0.8 | 0.8 | 0.9 | 1.0 | 1.4 | 1.4 | 1.3 | 1.3 |
| Mead's acid | 20:3w9 | 0.4 | 0.3 | 0.4 | 0.4 | 0.3 | 0.3 | 0.3 | 0.3 |
| Dihomogamma- | 20:3w6 | 2.4 | 2.3 | 2.3 | 2.4 | 2.0 | 1.9 | 1.9 | 1.9 |
| linolenic acid | | | | | | | | | |
| Arachidonic | 20:4w6 | 8.2 | 7.8 | 7.8 | 8.1 | 8.6 | 9.1 | 9.1 | 9.5 |
| | 20.3w3 | 0.0 | 0.1 | 0.1 | 0.1 | 0.0 | 0.0 | 0.0 | 0.0 |
| | 20:4w3 | 0.2 | 0.2 | 0.2 | 0.2 | 0.1 | 0.1 | 0.1 | 0.1 |
| Eicosapentaenoic | 20:5w3 | 2.5 | 2.6 | 2.4 | 2.5 | 2.3 | 2.3 | 2.2 | 2.2 |
| Behenic | 22:0 | 0.7 | 0.7 | 0.6 | 0.6 | 0.9 | 0.9 | 0.9 | 0.9 |
| Erucic | 22:1w9 | 0.3 | 0.3 | 0.2 | 0.2 | 0.3 | 0.2 | 0.2 | 0.2 |
| Docosatetraenoic | 22:4w6 | 2.4 | 2.3 | 2.5 | 2.5 | 1.9 | 2.0 | 2.1 | 2.1 |
| Docosapentaenoic- | 22:5w6 | 0.0 | 0.1 | 0.3 | 0.4 | 0.3 | 0.1 | 0.1 | 0.1 |
| omega 6 | | | | | | | | | |
| Docosapentaenocic- | 22:5w3 | 4.3 | 4.7 | 4.6 | 4.9 | 5.2 | 5.4 | 5.2 | 5.3 |
| omega 3 | | | | | | | | | |
| Lignoceric | 24:0 | 0.3 | 0.4 | 0.4 | 0.3 | 0.4 | 0.5 | 0.5 | 0.5 |
| Docosahexaenoic | 22:6w3 | 1.7 | 1.8 | 1.6 | 1.8 | 2.0 | 2.1 | 2.0 | 2.1 |
| Nervonic | 24:1 | 0.7 | 0.7 | 0.8 | 0.7 | 0.6 | 0.7 | 0.7 | 0.6 |
| | other | 5.6 | 5.5 | 6.3 | 5.9 | 7.3 | 6.0 | 7.6 | 7.9 |
| | sum | 100.0 | 100.0 | 100.0 | 100.0 | 100.0 | 100.0 | 100.0 | 100.0 |
| | Saturates | 36.7 | 36.6 | 38.1 | 37.9 | 40.7 | 40.6 | 40.5 | 40.2 |
| | Monoenes | 26.6 | 27.1 | 24.9 | 24.6 | 22.3 | 22.9 | 22.0 | 21.2 |
| | PUFA | 31.1 | 30.8 | 30.7 | 31.6 | 29.8 | 30.6 | 29.9 | 30.7 |
| | HUFA | 22.9 | 23.0 | 22.8 | 24.0 | 23.3 | 23.9 | 23.6 | 24.2 |
| | T/T | 0.0 | 0.0 | 0.0 | 0.0 | 0.0 | 0.0 | 0.0 | 0.0 |
| | Tot. w3 | 9.4 | 10.1 | 9.4 | 10.1 | 10.0 | 10.4 | 9.9 | 10.1 |
| | Tot. w6 | 21.3 | 20.4 | 20.9 | 21.1 | 19.5 | 19.9 | 19.7 | 20.3 |
| | Tot. w9 | 21.8 | 21.9 | 22.5 | 22.1 | 19.9 | 20.1 | 19.8 | 19.2 |
| | w6/w3 | 2.3 | 2.0 | 2.2 | 2.1 | 2.0 | 1.9 | 2.0 | 2.0 |
| | 20:4/20:5 | 3.3 | 2.9 | 3.2 | 3.2 | 3.8 | 3.9 | 4.2 | 4.3 |

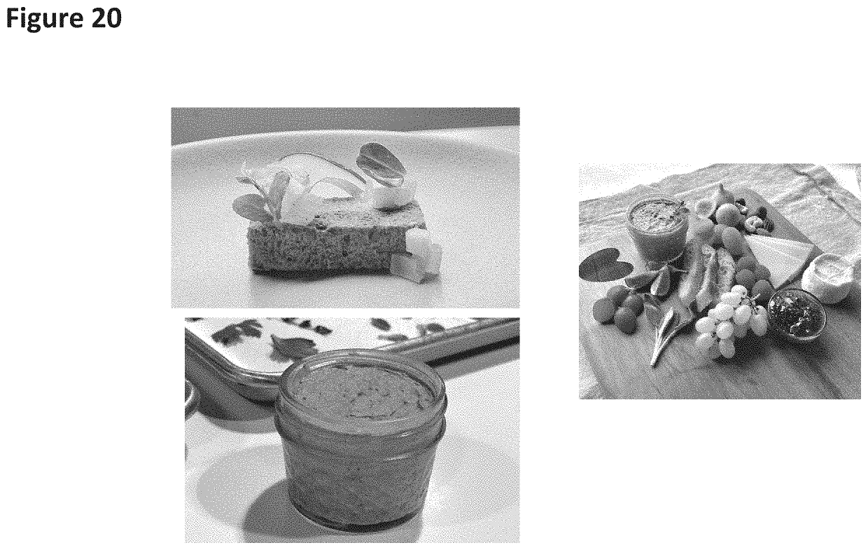

FIG. 20 shows a chicken pate prototype developed from embryonic chicken fibroblast transdifferentiated into hepatocytes by overexpression of HFN4a. The chicken pate prototype contains approximately 37.5% liver tissue, meeting the industry standards of at least 30% liver tissue for pate. See Labeling Policy Book at page 123. Additionally, the chicken pate prototype achieved a brown color and buttery texture similar to conventional pate.

The below Tables 5 and 6 depict the process of and ingredients employed for generation of chicken pate. This example is isolated and by no means limiting. As shown below, a tissue comprising non-transfected control hepatocytes was initially mixed with small amounts of red radish and brown carrot to develop color. The ingredients were weighed and combined. Forks were employed to make sure color was evenly added to the tissue. The color-treated tissue was then subjected to further processing to generate foie gras.

TABLE 5

| Ingredient | % | Actual (g) | 18 g | 37 g | |
|---|---|---|---|---|---|
| Hepatocytes | 99.6501749125 | 9.97 | 17.9370314843 | 36.8705647176 | 56.8005997001 |
| Red Raddish | 0.1499250375 | 0.015 | 0.0269865067 | 0.0554722639 | 0.0854572714 |
| Brown Carrot | 0.19990005 | 0.02 | 0.035982009 | 0.0739630185 | 0.1139430285 |
| Total | 100 | 10.005 | 18 | 37 | 57 |

TABLE 6

| Ingredient | % | Actual (g) | 50 g batch | |
|---|---|---|---|---|
| Tissue - Color | 37.5657400451 | 10 | 18.7828700225 | 56.3486100676 |
| Treatment 1 | | | | |
| Suspension, | 18.7828700225 | 5 | 9.3914350113 | 28.1743050338 |
| cooked | | | | |
| Butter | 18.7828700225 | 5 | 9.3914350113 | 28.1743050338 |
| Shallot | 5.6348610068 | 1.5 | 2.8174305034 | 8.4522915101 |
| Garlic | 3.7565740045 | 1 | 1.8782870023 | 5.6348610068 |
| Thyme, fresh, | 0.4507888805 | 0.12 | 0.2253944403 | 0.6761833208 |
| chopped | | | | |
| Port Wine | 11.2697220135 | 3 | 5.6348610068 | 16.9045830203 |
| Heavy Cream | 3.7565740045 | 1 | 1.8782870023 | 5.6348610068 |
| Salt, kosher | TT | TT | TT | TT |
| Black Pepper, | TT | TT | TT | TT |
| ground | | | | |
| Total | 100 | 26.62 | 50 | 150 |

In one particular, non-limiting example, foie gras was made by first pan melting butter on medium heat until foaming initiated. The color-treated tissue was then added until browned. Browning of the tissue required approximately three minutes of cooking on each side. Shallots, garlic, and thyme were then added, and the mixture further cooked for one minute. Port wine was then added to the mixture and reduced by half by cooking for approximately thirty seconds. The mixture was then blended in a food processor with butter, heavy cream, and cooked suspension until smooth. The blended mixture was seasoned with salt and pepper. The resulting mixture was then covered with plastic wrap and chilled for at least two hours or overnight to generate the final foie gras product. The foie gras could be served for consumption after thirty minutes at room temperature.

In some instances, the production of a foie gras or pate product using the cells of the present disclosure may include the additional step of supplementing the grown cells with additional fatty acids. Additional fatty acids may simply increase the overall concentration of fat in the end product, may be used to enhance the flavor of the end product, or some combination thereof. In one example, plant based lipids are blended or otherwise added to the cells as the cells are prepared into a food product to increase fatty acid concentration, to enhance flavor, or both.

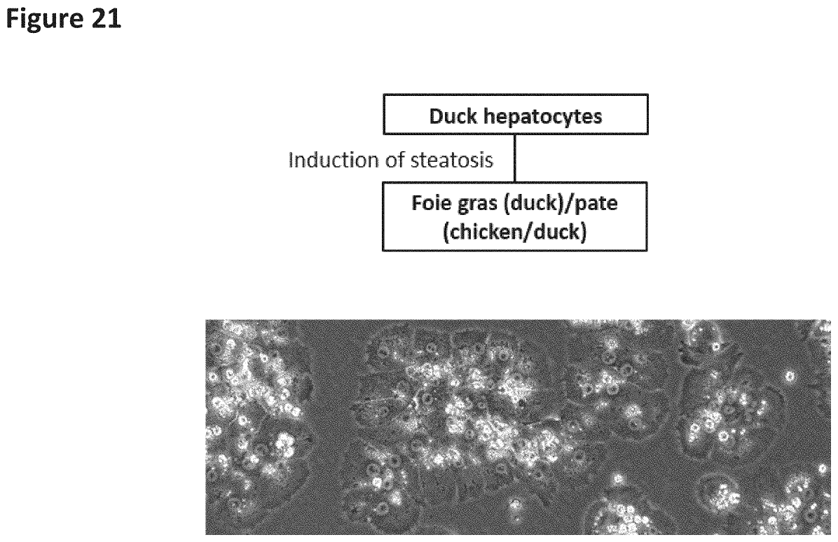

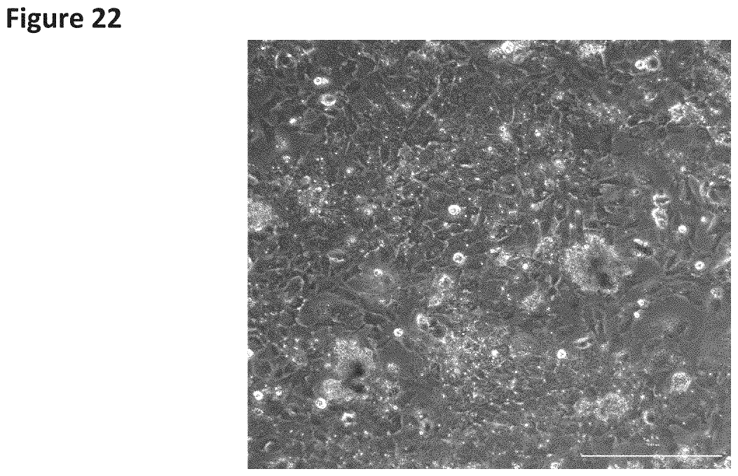

FIGS. 21 and 22 explore an alternative pathway for producing foie gras. Rather than transdifferentiating fibroblast into hepatocytes or adipocytes, this method starts by directly collecting hepatocytes from, for example, a duck. The hepatocytes are collected from either a juvenile or embryonic duck. The cells are isolated, passaged, and selected for high growth. Cells that are highly proliferative, survive suspension adaptation, and show a propensity to form tissue are then cultured and, once grown and collected, processed into a foie gras or pate product.

FIG. 21 provides a bright field microscopy view of primary duck hepatocytes collected from juvenile Pekin duck. These cells show intracellular lipid accumulation, which is a hallmark characteristic of liver cells.

FIG. 22 provides a bright field microscopy view of embryonic duck hepatocytes. These cells also show intracellular lipid accumulation, which is a hallmark characteristic of liver cells.

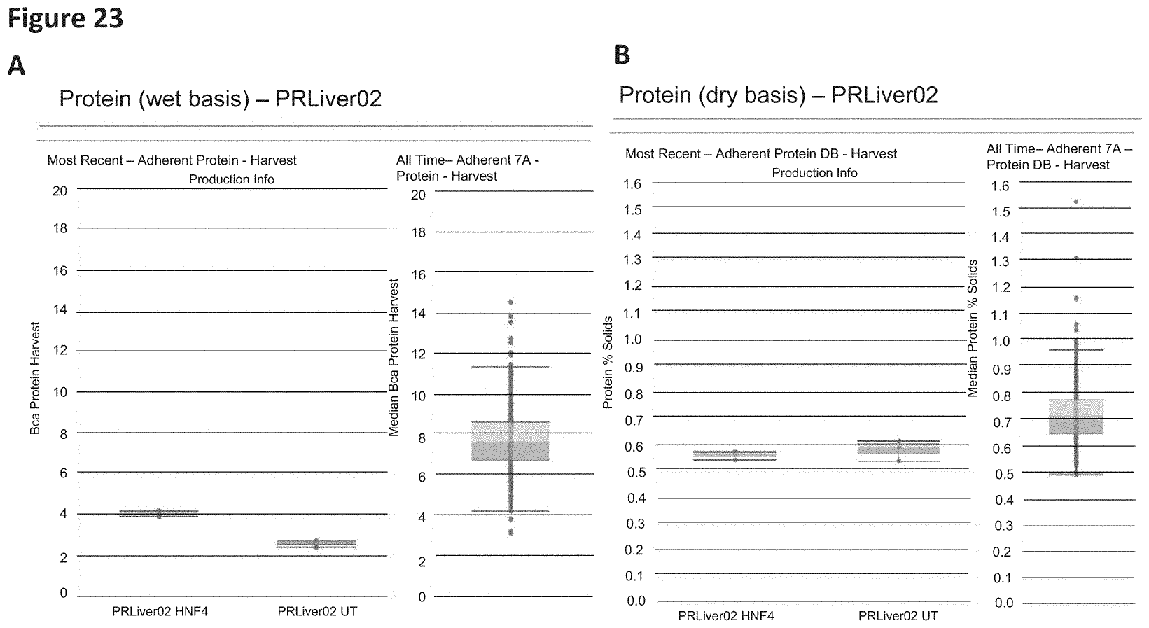

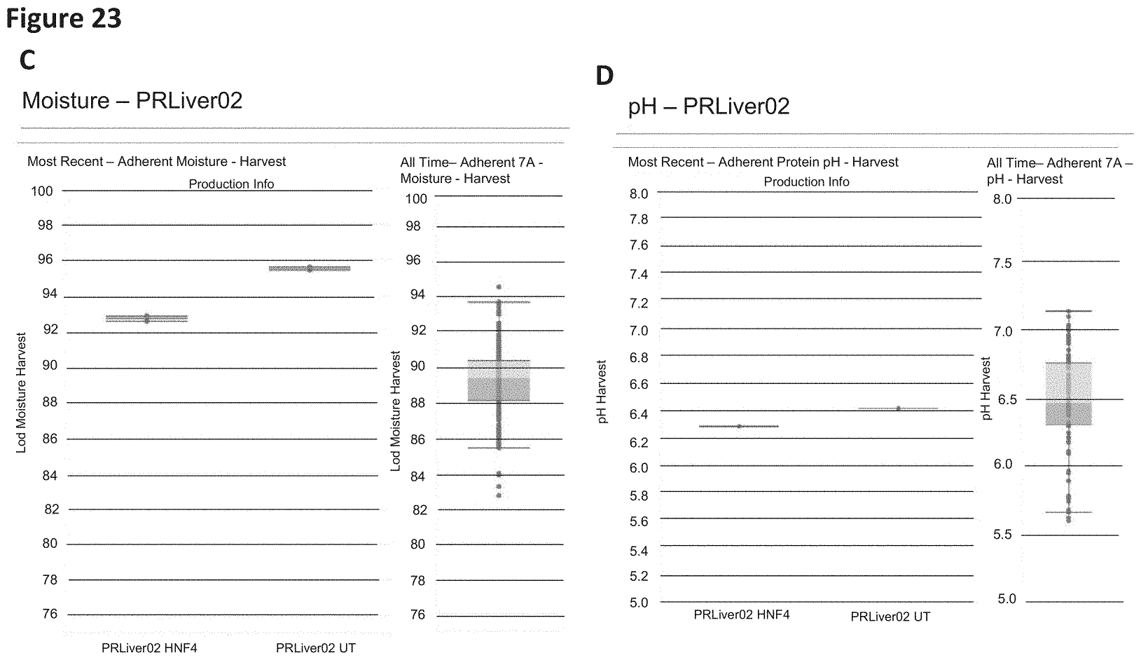

FIG. 23, Graphs A-D, compare protein levels, moisture content, and pH data, respectively, for fibroblasts versus liver tissue in HNF4alpha-transfected and untransfected samples. These data demonstrate that wet fibroblasts exhibit similar protein levels as wet liver tissue (FIG. 23, Graph A); dry fibroblasts exhibit similar protein levels as dry liver tissue (FIG. 23, Graph B); and moisture levels (FIG. 23, Graph C) and pH (FIG. 23, Graph D) are similar between fibroblasts and liver tissue.

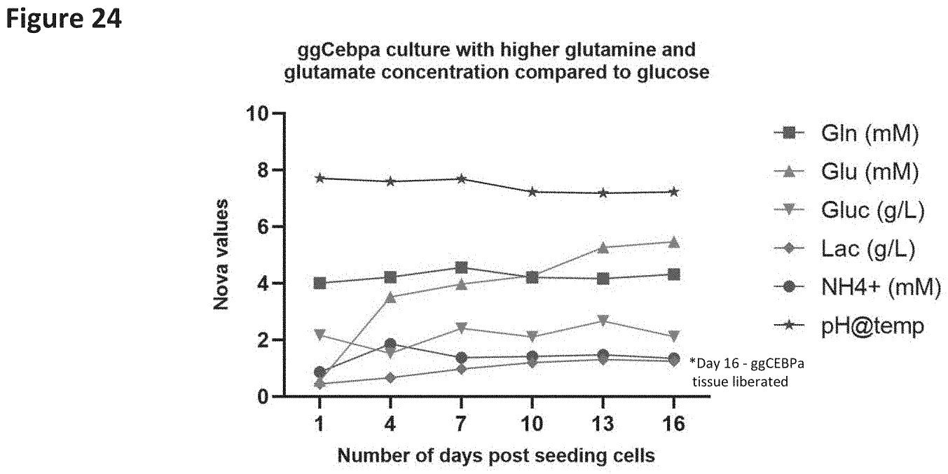

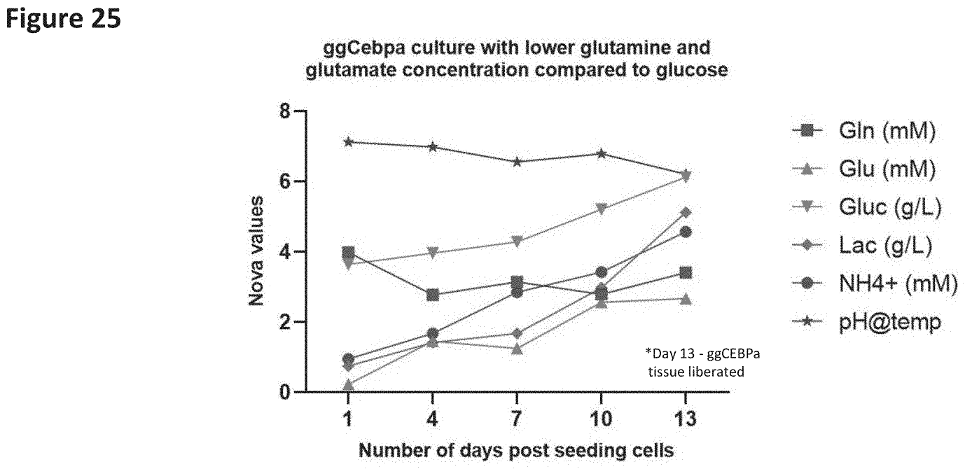

FIGS. 24 and 25 graphically illustrate a quantitative analysis of variations in cell culture media composition as ggCEBPa overexpressing cells grow and proliferate as a function of time. In some embodiments, the cell culture media is serum free or animal component free (ACF).To collect this data, ggCEBPa cells are first seeded using growth media and then flooded with concentrated metabolites, with the objective of reducing waste products and preventing early tissue liberation. When glucose is added into the cell culture at a lower concentration compared to glutamine and glutamate, ggCEBPa cells will consume higher levels of glutamine and glutamate compared to glucose, thereby keeping the amount of lactate and ammonia steady and below a value of 4, as illustrated. Steady levels of lactate and ammonium below a value of 4 causes the pH of the culture to remain in the normal range of 6.9-7.7. By altering the concentration of glucose, glutamine, and glutamate in this manner, waste products remained low and ggCEBPa tissue remained attached in culture plates for 16+ days. However, when glucose is added into the cell culture at a higher concentration compared to glutamine and glutamate, ggCEBPa cells will consume higher levels of glucose and will consequently produce increasing levels of lactate and ammonium as waste products. Increasing levels of lactate and ammonium causes the pH of the culture to decrease from a normal/healthy range of 6.9-7.7 to 6.2. This drop in pH and increase in waste products caused the ggCEBPa tissue to liberate on day 13.

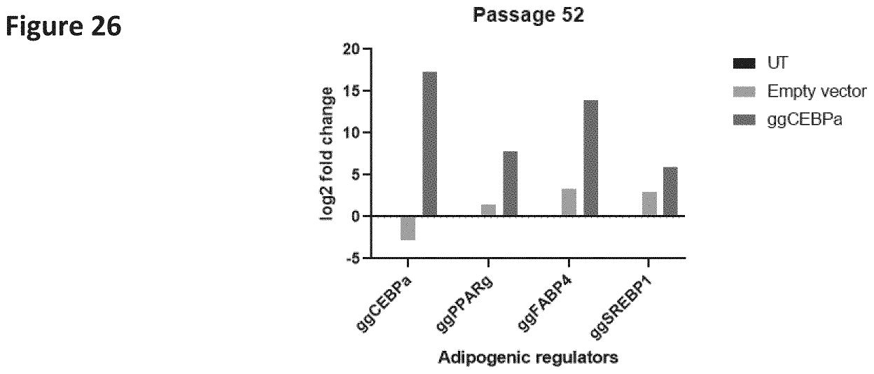

FIG. 26 graphically illustrates mRNA expression data for high passage ggCEBPa cells. In particular, these cells have been passaged 52 times, and are considered late passage cells. Cells of this advanced age typically have a reduction in differentiation and proliferation capacity. However, as detailed here, the cells of the present disclosure retain expression of not only the transfected C/EBPalpha gene, but they also retain expression of other genes in the adipocyte and lipid generation pathway (i.e. adipogenic factors). Expression such as this indicates robust stability of the adipocyte phenotype, which is remarkable for such late passage cells. Furthermore, this stability is particularly advantageous for large scale production of cell based meat, because such stability ensures continuity and consistency among all products developed from cells descending from this cell line. To generate this data, passage 52 was reached by passaging the ggCEBPa cells every 3 days, which is typically when the cells have reached a confluence of about 80-90%. Y-axis log 2 fold change are the units for mRNA expression determined with RT-qPCT. A “passage” is when cells are detached from culture plate using Trypsin, the Trypsin is then neutralized with culture media, the cells are counted, and a % of the viable cells are pipetted from the original cell culture plate to the new culture plate. Fresh culture media is added to the culture with cells. This process indicates 1 passage. The cells are passaged to a new culture plate when having reached a confluence of about 80-90%. PDL refers to “population doubling level”. This number indicates how many times the cells have doubled in population over a period of time. If, for example, over a 24 hour time period the cells have doubled twice since the cells were thawed after cryopreservation, then the cells have a PDL of 2.

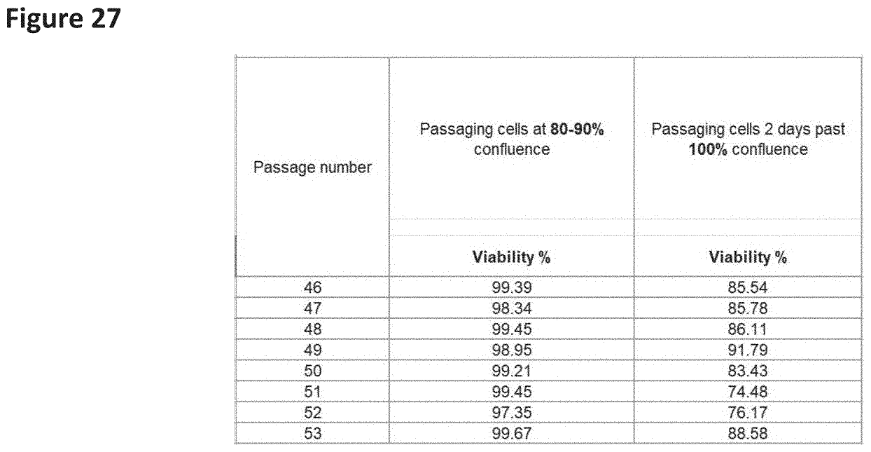

FIG. 27 illustrates two separate cell passaging techniques and the resulting cell viability percentage from each technique. Cells overexpressing ggCEBPa were passaged seven times, i.e. from passage 46 to passage 53, using two separate methods. In the first method, cells were passaged when the cells reached about 80-90% confluence. In the second method, cells were passaged two days after the cells had reached complete (i.e. 100%) confluence. As demonstrated by FIG. 27, passaging ggCEBPa cells when the cells have reached about 80-90% confluence leads to consistently higher cell viability as compared to passaging ggCEBPa cells after they have already reached confluence. In particular, when the cells are left in culture to reach 100% confluence viability decreases by about 8-25%. Thus, increased efficiency of cell passaging can be achieved by passaging cells when the culture reaches about 80-90% confluence.

### REFERENCES

All references referred to above are incorporated herein by reference in their entireties.

What is claimed is:

1. An in-vitro cultured meat product, comprising: a population of cells comprising fibroblasts, myoblasts, or a combination thereof, the population of cells being transdifferentiated to express adipocyte phenotypes; wherein the transdifferentiation involves transfection to induce steatosis via an overexpression of CEPBalpha, CEPBgamma, PPARgamma, SREBP1, SREBP2 or a combination thereof.

2. The in-vitro cultured meat product of claim 1, wherein the population of cells is transfected to downregulate at least one of OSR1, PRRX1, LHX9, TWIST2, and INSIG2.

3. The in-vitro cultured meat product of claim 2, wherein transdifferentiation occurs without endogenous hormones or small molecules recognized to transdifferentiate cells into adipocyte phenotypes.

4. The in-vitro cultured meat product of claim 3, wherein the transdifferentiated population of cells retains proliferative capacity at late passage.

5. The in-vitro cultured meat product of claim 3, wherein the transdifferentiated population of cells exhibits a stable phenotype at late passage.

6. The in-vitro cultured meat product of claim 1, wherein the population of cells is transfected to overexpress HNF4alpha.

7. The in-vitro cultured meat product of claim 1, wherein the cells are transdifferentiated to express hepatocyte phenotypes.

8. The in-vitro cultured meat product of claim 1, wherein the population of cells includes myoblasts having wildtype MyoD.

9. The in-vitro cultured meat product of claim 8, wherein the wildtype MyoD is overexpressed.

10. The in-vitro cultured meat product of claim 1, wherein the transdifferentiated population of cells exhibits lipid droplet formation in the cytoplasm.

11. An in-vitro cultured meat product, comprising:
a. 50-95% in-vitro cultured meat by weight, wherein the in-vitro cultured meat comprises a population of cells transdifferentiated to express adipocyte phenotypes, a population of cells transdifferentiated to express at least one of hepatocyte phenotypes, adipocyte lineage cells, hepatocyte lineage cells, or a combination thereof; and
b. 5-19% butter, cream, or a combination thereof, by weight.

12. The in-vitro cultured meat product of claim 11, wherein the butter and/or cream are replaced by a plant-based lipid alternative.

13. The in-vitro cultured meat product of claim 12, wherein the plant-based lipid alternative is a natural oil, canola, vegetable oil, safflower oil, margarine, or some combination thereof.

14. The in-vitro cultured meat product of claim 11, comprising one or more of radishes and carrots at 0.1% to 1% by weight.

15. The in-vitro cultured meat product of claim 11, comprising one or more of shallots, garlic, and thyme at 0.5% to 6% by weight.

16. The in-vitro cultured meat product of claim 11, comprising 1-12% port wine by weight.

17. The in-vitro cultured meat product of claim 16, wherein the port wine is reduced.

18. The in-vitro cultured meat product of claim 11, wherein the population of cells is transfected to overexpress at least one of HNF4alpha, liver lineage cells, or some combination thereof.

19. The in-vitro cultured meat product of claim 11, wherein the in-vitro cultured meat comprises a population of cells transfected to induce steatosis via an overexpression of CEPBalpha, CEPBgamma, or some combination thereof.

20. A method of cooking an in-vitro cultured meat product, comprising:
a. melting a lipid in a cooking apparatus;
b. adding in-vitro cultured meat to the cooking apparatus, wherein the in-vitro cultured meat comprises a population of cells transdifferentiated to express adipocyte phenotypes; and
c. cooking at least one side of the in-vitro cultured meat product until a color change or texture change occurs.

21. The method of claim 20, wherein the in-vitro cultured meat comprises a population of cells transfected to induce steatosis via an overexpression of CEPBalpha, CEPBgamma, or a combination thereof.

22. The method of claim 20, further comprising:
a. adding one or more of shallots, garlic, thyme, port wine, salt, and pepper to the in-vitro cultured meat product; and
b. blending the in-vitro cultured meat product until smooth with the lipid;

23. The method of claim 22, further comprising cooling the in-vitro cultured meat product until chilled.

24. The method of claim 20, wherein the lipid is replaced with plant-based alternatives.

25. The method of claim 24, wherein the plant-based lipid alternatives comprise natural oil, canola, vegetable oil, safflower oil, margarine, or some combination thereof.
